(19) United States
(12) Patent Application Publication
(10) Pub. No.: US 2024/0245834 A1
(43) Pub. Date: Jul. 25, 2024

(54) DRUG-ELUTING TUBES FOR IMPROVED PATIENT CARE

(71) Applicant: BOARD OF REGENTS, THE UNIVERSITY OF TEXAS SYSTEM, Austin, TX (US)

(72) Inventors: Daniel Stromberg, Austin, TX (US); Aaron Baker, Austin, TX (US); Austin Veith, Austin, TX (US)

(21) Appl. No.: 18/566,362

(22) PCT Filed: Jun. 2, 2022
(86) PCT No.: PCT/US2022/031953

§ 371 Date: Dec. 1, 2023

Related U.S. Application Data
(60) Provisional application No. 63/195,832, filed on Jun. 2, 2021.

(51) Int. Cl. A61L 29/16; A61K 9/00; A61K 31/445; A61L 29/06; A61L 29/12; A61M 25/00; A61M 27/00

(52) U.S. Cl. CPC A61L 29/16; A61K 9/0024; A61K 31/445; A61L 29/06; A61L 29/126; A61M 27/00; A61L 2300/402; A61L 2300/416; A61L 2300/606; A61L 2300/622; A61L 2400/10; A61M 2025/0046; A61M 25/0054; A61M 25/007

(57) ABSTRACT

The present invention provides improved drug-eluting tube devices. In some embodiments, the tube devices deliver analgesics directly at sites of internal discomfort, including, but not limited to, internal membranes and implant entry sites. In some embodiments, the tube devices can be reloaded with drug in situ. In some embodiments, the tube devices comprise lubricating coatings to reduce painful friction with internal membranes. In some embodiments, the tube devices further include flexible and pliable regions that dampen force transmission to implanted sites, such as by inadvertent pulling.

### CROSS-REFERENCE TO RELATED APPLICATIONS

This application claims priority to and the benefit of U.S. Provisional Application No. 63/195,832, filed Jun. 2, 2021, the disclosure of which is incorporated herein by reference in its entirety.

### BACKGROUND OF THE INVENTION

Patients who undergo surgical interventions often require drainage tubes in the postoperative period to avoid dangerous accumulation of fluids or gases. The tubes are typically kept in place for several days while healing ensues and drainage dissipates. Although the tubes are made of softened plastics, patients universally complain of pain and irritation at the internal sites in contact with the tubes and at the chest wall entry site. Currently, strong systemic pain medications (predominantly narcotics) are given for analgesia with variable effectiveness, high cost, and many attendant complications (e.g., addiction, respiratory depression, hemodynamic instability, slowed intestinal function, urinary retention, excessive sedation, etc.). In addition, many patients who have chest tubes or catheters are fearful of the pain caused by accidental tugging on their devices through the normal course of hospital activities or patient motion.

Thus, there is a need in the art for improved tube devices and implants that address associated mechanical and sensory discomforts. The present invention meets this need.

### SUMMARY OF THE INVENTION

In one aspect, the present invention provides a tube device comprising: an elongate tube body having an exterior surface, an interior surface, and at least one lumen extending between a proximal end and a distal end; and at least one drug loaded on the tube body.

In one embodiment, the tube body is porous, such that the at least one drug is loaded within pores of the porous tube body. In one embodiment, the at least one drug is encased or embedded in a microparticle. In one embodiment, the microparticle comprises a poly(lactide-co-glycolides) (PLGA). In some embodiments, the PLGA is selected from 75:25 PLGA, 70:30 PLGA, 65:35 PLGA, 60:40 PLGA, or 50:50 PLGA. In one embodiment, the at least one drug is loaded with a desiccated or lyophilized solution within the pores. In one embodiment, the at least one drug is loaded in a drug-release polymer within the pores following removal of at least one solvent. In one embodiment, the tube body is reloadable with the at least one drug by occluding the at least one lumen and introducing the at least one drug in a solution into the at least one lumen under a positive pressure such that the at least one drug is filtered by the porous tube body. In one embodiment, the at least one drug is loaded in a drug-eluting coating positioned on the exterior surface.

In one embodiment, the tube body comprises at least one flexible region positioned between the proximal end and the distal end. In one embodiment, the tube body has a length between about 10 cm to about 100 cm. In one embodiment, the tube body has an outer diameter between about 1 mm (3 Fr) to about 11.3 mm (34 Fr). In one embodiment, the tube body has an inner diameter between about 0.5 mm to about 10 mm. In one embodiment, the tube body comprises one or more apertures fluidly connected to the at least one lumen.

In one embodiment, the drug-eluting coating comprises a desiccated/lyophilized or hydrated gel composition. In one embodiment, the drug-eluting coating is a lubricated coating.

In one embodiment, the at least one drug comprises one or more anesthetic selected from the group consisting of: articaine, benzocaine, benzonatate, bupivacaine, chloroprocaine, cinchocaine, diclofenac-diethylamine, dimethocaine, eucaine, etidocaine, exparel, hexylcaine, levobupivacaine, lidocaine, mepivacaine, meprylcaine, oxybuprocaine, phenacaine, piperocaine, pramocaine, prilocaine, procaine, proparacaine, quinisocaine, ropivacaine, tetracaine, and trimecaine.

In one embodiment, the at least one drug comprises one or more adjuvant selected from the group consisting of: epinephrine, narcotics, steroids, clonidine, dexmedetomidine, parecoxib, lornoxicam, midazolam, ketamine, magnesium sulfate, neostigmine, tonicaine, n-butyl tetracaine, adenosine, dextran, neuromuscular blocker, and butamben.

In one embodiment, the at least one drug comprises a therapeutic selected from the group consisting of: anti-viral agents, anti-bacterial agents, anti-thrombolytics, anti-fibrinolytics, chemotherapeutics, anti-hypertensives, immunotherapeutics, antibiotics, anti-inflammatory agents, pro-inflammatory agents, antiseptics, anesthetics, analgesics, pharmaceutical agents, antibodies, small molecules, peptides, and nucleic acids.

In one embodiment, the tube body further comprises one or more reservoir or track, such that the at least one drug is stored in the one or more reservoir or track. In one embodiment, the tube device further comprises a protective sheath encasing at least a portion of the tube body. In one embodiment, the at least one flexible region comprises one or more springs or corrugations. In one embodiment, the at least one flexible region comprises a section of the tube body constructed from a flexible and stretchable membrane. In one embodiment, the at least one flexible region comprises alternating flexible sections and stiff sections. In one embodiment, the flexible sections comprise a flexible and stretchable membrane. In one embodiment, the stiff sections comprise a section of the tube body. In one embodiment, the flexible sections are supplemented with one or more springs.

In one embodiment, the elongate tube body comprises ultra-high-molecular-weight polyethylene.

In one embodiment, the elongate tube body is constructed from a flexible and stretchable membrane. In one embodiment, the flexible and stretchable membrane comprises a silicone. In one embodiment, the elongate tube body is a silicone tube.

In one embodiment, the tube device forms part of a medical device selected from the group consisting of: chest tubes, endotracheal tubes, Foley catheters, surgical drains, prosthetics, orthopedic implants, and breast implants.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1 depicts a perspective view of an exemplary tube device.

FIG. 2 depicts a schematic of an exemplary porous tube device loaded with a therapeutic.

FIG. 3A through FIG. 3C depict a side profile of an experimental porous tube device (FIG. 3A, top), a loading setup of the porous tube device (FIG. 3A, bottom), and reloading setups of the porous tube device (FIG. 3B, FIG. 3C).

FIG. 4 depicts a schematic of another exemplary porous tube device loaded with a therapeutic.

FIG. 5 depicts a schematic of an exemplary surface-modified tube device.

FIG. 6 depicts perspective and cross-sectional views of exemplary tube devices.

FIG. 7A and FIG. 7B depict cross-sectional views of further exemplary tube devices and drug delivery means. FIG. 7A depicts cross-sectional views of exemplary tube devices and drug delivery means comprising an adherent. FIG. 7B depicts cross-sectional views of exemplary tube devices and drug delivery means that do not comprise an adherent.

FIG. 8 depicts perspective views of exemplary tube device flexible regions.

FIG. 9 depicts perspective views of further exemplary tube device flexible regions.

FIG. 10 depicts SEM images of prototype tube devices showing the difference in grain structure of ultra-high molecular weight polyethylene (UHMWPE) tubes. The 15-20 throat radius tube (UHMWPE 15 also referred to as Sintered Tube 1 (ST1), right) has a much tighter grain and pore structure than the 30-40 throat radius tube (UHMWPE 30 also referred to as Sintered Tube 2 (ST2), middle). SEM image of a third UHMWPE prototype tube device Sintered Tube 3 (ST3) is also shown (left). Throat radius refers to connections between pores and indicates the size of spaces that microparticles flow through.

FIG. 11 depicts the results of pore analysis of a section of a UHMWPE 15 catheter also referred to as ST1. The porosity of the section was about 33.6%, with an average pore radius of 60.6 μm and an average throat radius of about 18.7 μm.

FIG. 12 depicts the results of pore analysis of a section of a UHMWPE 30 catheter also referred to as ST2. The porosity of the section was about 37.1%, with an average pore radius of 82.1 μm and an average throat radius of about 29.6 μm.

FIG. 13 depicts the results of pore analysis of a section of a ST3.

FIG. 14 depicts representative results demonstrating the mechanical properties of ST1, ST2, ST3, and control polyvinylchloride (PVC) prototype tube devices.

FIG. 15 depicts the results of experiments demonstrating drug loading capacity of microparticles (left) and release of drug from the microparticles in a UHMWPE 15 catheter (also referred to as ST1, right). The microparticle loading capacity is a measurement of the percent of the mass of a microparticle that is composed of a drug (here, bupivacaine). The graph shows at least 40% of the total mass of the microparticle formulations is bupivacaine. The cumulative release of bupivacaine is shown over the course of a 14 day period.

FIG. 16 depicts the results of experiments investigating bupivacaine standards spectra as well as a standard curve generated from the bupivacaine standards. The ultraviolet-visible (UV-vis) absorbance spectra are shown for each of the concentrations (from 1000 μg/mL to 31.25 μg/mL, left). The standard curve is used to convert UV-vis absorbance to bupivacaine concentrations. The maximum absorbance peak for bupivacaine occurs at 262 nm. The value of the spectra at this point is used to generate the standard curve (right). The inset on the spectra graph shows the peak that occurs at 262 nm.

FIG. 17 shows a sample of recorded spectra wherein the measured signal has a similar morphology to the pure bupivacaine signal measured in the standard curves, demonstrating that the measured spectra is in fact bupivacaine released from the tubes.

FIG. 18 shows a release curve for a pilot tube having a 65:25 25% bupivacaine formulation in a UHMWPE 15 tube (also referred to as ST1). Cumulative drug release is shown over the course of 27 days.

FIG. 19 shows the results of cumulative drug release curves from different formulations of bupivacaine in UHMWPE 15 tubes (also referred to as ST1) over the course of 21 days (top), UWMWPE 30 tubes (also referred to as ST2) over the course of 10 days (bottom), and ST3 over the course of 21 days. All graphs are ratioed by length and adjusted to represent release per centimeter of tube. The curves are plotted in two ways. The first is the total bupivacaine released into solution in μg. The second is in terms of the amount of bupivacaine released as a percentage of the total amount loaded into a respective tube at the beginning of the study.

FIG. 20 depicts a porous tube fabricated from PVC (left) and an SEM image of the porous PVC tube (right).

FIG. 21 depicts the results of pore analysis of a section of a porous PVC catheter. The porosity of the section was about 24.2%, with an average pore radius of 49.0 μm and an average throat radius of about 13.8 μm.

FIG. 22 depicts the results of high performance liquid chromatography (HPLC) data from the release of a porous PVC catheter loaded with 75:25 PLGA microparticles. The y-axis is the cumulative release of bupivacaine in μg. These results demonstrate that bupivacaine release peaks at 12 hours, after which it falls due to the sampling that occurs during the release study.

FIG. 23 depicts the results of an amination reaction with PVC tubes. Groups tested were as follows: 4, 8, 16, and 24 hours in ethylene diamine (EDA) and a PVC control. The photo shows tubes in water after amination demonstrating the color change following incubation in EDA. After tubes were aminated, free amines on the tube surfaces were reacted with fluorescein isothiocyanate (FITC) to check for functionality. Sections of the tubes were scanned on a plate reader for FITC and the intensities plotted in the graph shown. The results show that maximum intensity is attained in the 8 hour incubation, demonstrating that this group has the highest number of amines on the surface of the tube.

FIG. 24 depicts the results of x-ray photoelectron spectroscopy (XPS) analysis performed on PVC tubes after amination. The top left image shows the tubes sections arranged in an XPS. The three graphs show the results of surface moiety analysis. Nitrogen is and carbon is peak areas were compared for each tube, with the results demonstrating that the maximum presence of surface nitrogen groups occurs in the 8 hour incubation, confirming that a maximum number of amine groups are present on the tube surface after 8 hours incubation in EDA. Chlorine content was analyzed for each tube and an increased presence of chlorine was found in every tube exposed to EDA, demonstrating that the modification reaction released chlorine ions in the form of hydrochloric acid. Sulfur content was analyzed for each tube demonstrating no significant change between the groups.

FIG. 25 depicts the results of reacting the surface aminated PVC tubes with glutaraldehyde. The photo shows tube sections after modification. The graph shows the results of a glutaraldehyde colorimetric assay. The results demonstrate that the 16 hour EDA aminated tubes have the most glutaraldehyde on the surface after modification.

FIG. 26 depicts the results of XPS analysis of PVC tubes after glutaraldehyde modification. After modification, the glutaraldehyde groups on the tube surfaces were separately reacted with molecules containing sulfur and molecules containing nitrogen. Higher levels of detected nitrogen and sulfur indicate that more glutaraldehyde is present on the tube surface. Here, the 8 hour incubation group demonstrated the highest level of amine groups and glutaraldehyde.

FIG. 27 depicts the results of mechanical characterization of aminated tubes. 3 mm sections of tubes were subjected to a 1 mm ring compression, with the force required to achieve the 1 mm displacement recorded (top left). The results demonstrate that longer exposure to EDA led to a greater amount of force required to produce the displacement (top right). Stiffness was calculated and compared to a standard chest tube. The results demonstrate that tubes exposed to EDA for 4 and 8 hours have 2× and 3× increased stiffness over the standard chest tube, respectively (bottom left).

FIG. 28 depicts cross-sectional views of exemplary tube devices and drug delivery means that comprise a silicone inner tube and drug eluting outer tube.

### DETAILED DESCRIPTION

The present invention provides improved drug-eluting tube devices. In some embodiments, the tube devices deliver analgesics directly at sites of internal discomfort, including, but not limited to, internal membranes and implant entry sites. In some embodiments, the tube devices can be reloaded with drug in situ. In some embodiments, the tube devices comprise lubricating coatings to reduce painful friction with internal membranes. In some embodiments, the tube devices further include flexible and pliable regions that dampen force transmission to implanted sites, such as by inadvertent pulling.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, +5%, +1%, and ±0.1% from the specified value, as such variations are appropriate.

“Anesthetic” as used herein refers to an agent that produces a reversible loss of sensation in an area of a subject's body.

“Lumen” as used herein refers to a canal, duct or cavity within a tubular structure.

As used herein, the term “composition” or “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism.

“Infusion” as used herein refers to a process of slow introduction of an element, for example a solution, into or onto a target.

As used herein, “nanoparticles” are particles generally in the nanoscale. Different morphologies are possible depending on the nanoparticle composition. It is not necessary that each nanoparticle be uniform in size. “Nanoparticles” encompass nanospheres, nanoreefs, nanorods, nanoboxes, nanocubes, nanostars, nanoshards, nanotubes, nanocups, nanodiscs, nanodots, quantum dots, and the like. They may be intrinsic particles or coated with bioactive ligands.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

A “therapeutic” treatment is a treatment administered to a subject who exhibits signs or symptoms of pathology, for the purpose of diminishing or eliminating those signs or symptoms.

“Track” as used herein refers to a conduit, duct or any type of longitudinal hollow path-way used for transport in either longitudinal direction. For example a track may be used for the delivery of an anesthetic agent, drug, or therapeutic down the track to target an anatomical site.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell line from a patient (e.g., for diagnosis or ex vivo applications), who has a condition contemplated herein, a symptom of a condition contemplated herein or the potential to develop a condition contemplated herein, with the purpose to cure, heal, alleviate, relieve, alter, remedy, ameliorate, improve or affect a condition contemplated herein, the symptoms of a condition contemplated herein or the potential to develop a condition contemplated herein. Such treatments may be specifically tailored or modified, based on knowledge obtained from the field of pharmacogenomics.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Tube Devices and Implants

The present invention provides coated tube devices and implants that elute drugs or therapeutics to provide patients with relief from pain associated with temporary and permanent implants, reduce the need for narcotic/systemic pain medication administration and related deleterious side effects, and decrease costs through complication avoidance and more efficient recovery. In addition, in certain embodiments, the coatings can include drugs or therapeutics with additional activities of interest, including antibiotics, anti-proliferative drugs, anti-inflammatory drugs, anti-thrombotics, etc. The tube devices and implants also prevent transmission of forces from the common occurrence of inadvertent tugging during routine patient care by containing flexible elements, such as elastic, accordion-like regions, or spring elements.

In some embodiments, the tube devices and implants comprise between about 0.01 μg to about 100 g of the drugs or therapeutics. In some embodiments, the tube devices and implants comprise between about 0.01 μg to about 100 mg of the drugs or therapeutics. In some embodiments, the tube devices and implants comprise between about 0.01 μg to about 50 mg of the drugs or therapeutics. In some embodiments, the tube devices and implants comprise between about 0.1 μg to about 10 mg of the drugs or therapeutics. In some embodiments, the tube devices and implants comprise between about 1 μg to about 5 mg of the drugs or therapeutics. In some embodiments, the tube devices and implants comprise between about 0.005 mg to about 80 mg of the drugs or therapeutics. In some embodiments, the tube devices and implants comprise between about 0.005 mg to about 50 mg of the drugs or therapeutics. In some embodiments, the tube devices and implants comprise between about 0.005 mg to about 30 mg of the drugs or therapeutics. In some embodiments, the tube devices and implants comprise between about 0.005 mg to about 25 mg of the drugs or therapeutics.

In some embodiments, the tube devices and implants deliver a dose of from about 0.001 ng/kg/day and about 100 mg/kg/day to the patient. For example, in some embodiments, the tube devices and implants deliver a dose of from about 0.005 mg/kg/day and about 5 mg/kg/day.

In some embodiments, the tube devices and implants deliver drugs or therapeutics dosage that ranges in amount from 0.001 μg to about 500 mg per kilogram of body weight of the patient, while the precise dosage administered will vary depending upon any number of factors, including but not limited to, the type of patient and type of disease state being treated, the age of the patient and the route of administration. Preferably, the dosage of the drugs or therapeutics will vary from about 0.01 μg to about 100 mg per kilogram of body weight of the patient. More preferably, the dosage will vary from about 1 μg to about 50 mg per kilogram of body weight of the patient. For example, in some embodiments, the dosage will vary from about 0.005 mg to about 5 mg per kilogram of body weight of the patient.

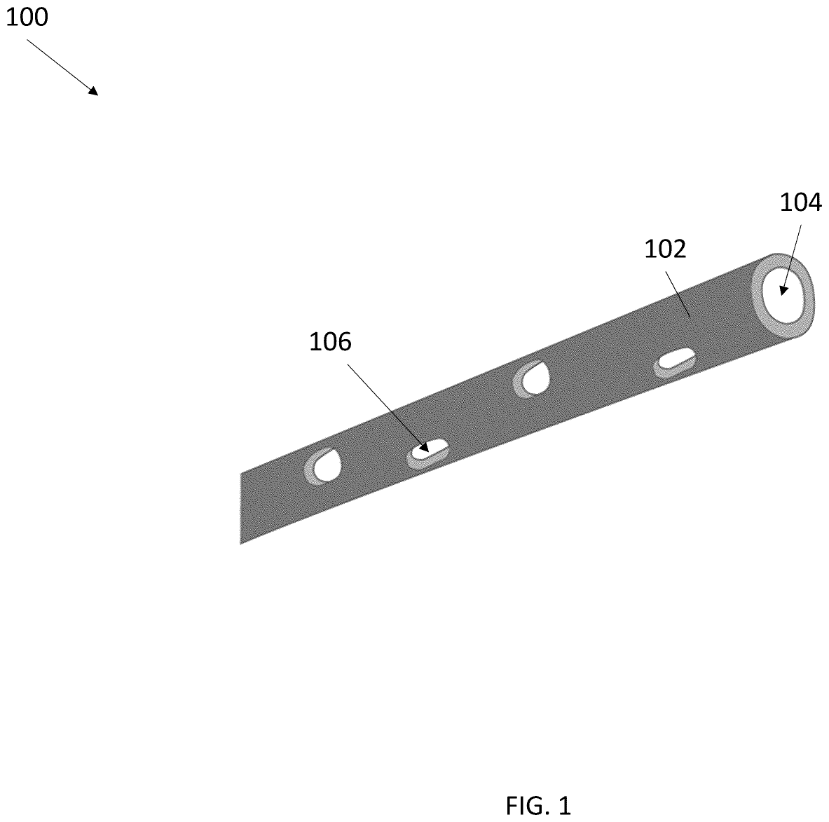

Referring now to FIGS. 1-9 and 28, exemplary tube devices 100 are depicted. Device 100 comprises tube body 102 having at least one lumen 104. Tube body 102 has an elongate tubular shape extending between a proximal end and a distal end. Tube body 102 can have any suitable dimensions, including, but not limited to, a length between about 10 cm to about 100 cm or more, an outer diameter between about 1 mm (3 Fr) to about 11.3 mm (34 Fr) or more, and an inner diameter between about 0.5 mm to about 10 mm or more. In some embodiments, device 100 comprises one or more apertures 106 embedded in tube body 102, each aperture 106 being fluidly connected to the at least one lumen 104.

Tube body 102 can be fabricated from any suitable material. Contemplated materials include biocompatible polymers, including, but not limited to, poly(urethanes), poly(siloxanes) or silicones, poly(ethylene), low density polyethylene (LDPE), high density polyethylene (HDPE), ultra-high molecular weight polyethylene (UHMWPE), high-density polyethylene (HDPE), cross-linked polyethylene (PEX or XLPE), medium-density polyethylene (MDPE), low-density polyethylene (LDPE), linear low-density polyethylene (LLDPE), very-low-density polyethylene (VLDPE), polytetrafluoroethylene (PTFE), poly(vinyl pyrrolidone), poly(2-hydroxyethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), poly(vinyl chloride) (PVC), polyvinylhydroxide, poly(ethylene oxide) (PEO), polyorthoesters, polysebacates, poly(glycerolsebacates), and the like. For example, in some embodiments, the PLGA comprises any PLGA known in the art, including, but not limited to, 99:1 PLGA, 95:5 PLGA, 90:10 PLGA, 85:15 PLGA, 80:20 PLGA, 75:25 PLGA, 70:30 PLGA, 65:35 PLGA, 60:40 PLGA, 55:45 PLGA, 50:50 PLGA, 45:55 PLGA, 40:60 PLGA, 35:65 PLGA, 30:70 PLGA, 25:75 PLGA, 20:80 PLGA, 15:85 PLGA, 10:90 PLGA, 5:95 PLGA, and/or 1:99 PLGA.

Tube body 102 can be fabricated using any suitable methods known in the art. Exemplary methods for fabricating tube body 102 include, but are not limited to, sintering, electrospinning, salt extraction, foaming, ion/electron beam treatment, woven/knitted materials, 3D printing, 3D sintering, or any combination thereof. For example, in some embodiments, tube body 102 is prepared using sintering.

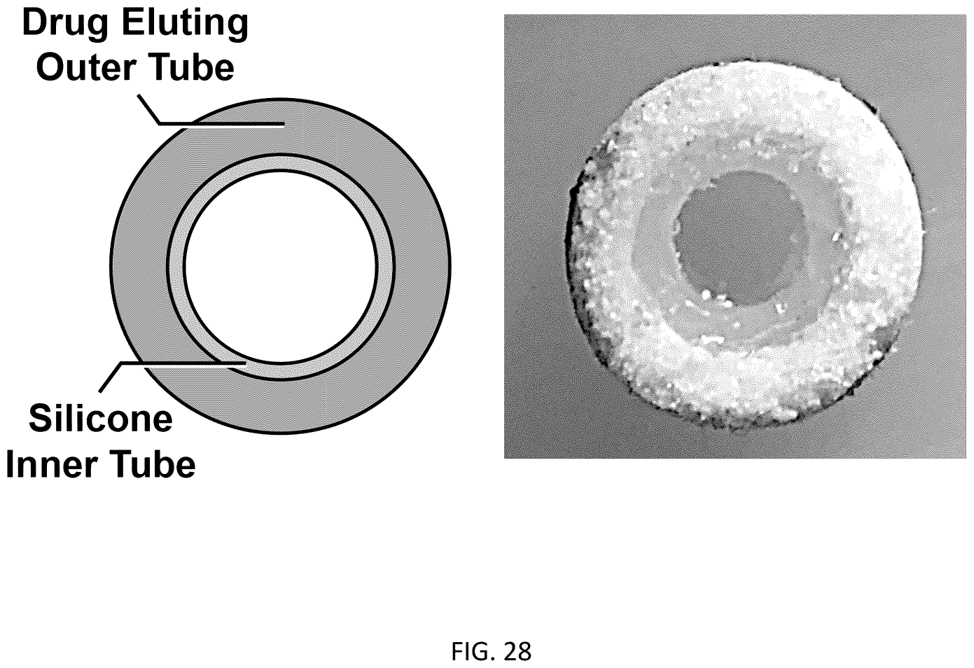

In some embodiments, device 100 comprises an outer drug eluting tube and an inner tube. In certain embodiments, the inner tube is non-porous (FIG. 28). For example, in certain embodiments, the non-porous inner tube minimizes or eliminates the undesirable loss of drug from the drug eluting outer tube that may otherwise occur during suction. In certain embodiments, the non-porous inner tube prevents loss of suction.

The inner tube can comprise any suitable material, including but not limited to, silicone. In one embodiment, the inner tube has a thickness of about 100 μm-2 mm. In one embodiment, the inner tube has a thickness of about 250 μm-500 μm.

In some embodiments, tube body 102 comprises a porous construction and a non-porous construction. For example, in some embodiments, tube body 102 comprises a porous construction and a non-porous lining. In some embodiments, tube body 102 comprises a porous exterior surface and a non-porous interior surface. For example, in some embodiments, tube body 102 comprises a porous exterior surface and a silicone interior surface. In some embodiments, the non-porous interior surface prevents loss of suction. In some embodiments, the non-porous interior surface minimizes or eliminates an undesirable drug removal. In some embodiments, the porous exterior surface is a porous exterior tube. In some embodiments, the non-porous interior surface is a non-porous interior tube. Thus, in some embodiments, tube body 102 comprises a porous exterior tube and a non-porous interior tube. For example, in some embodiments, tube body 102 comprises a porous exterior tube and a silicone interior tube.

### Microparticle Embedded Device

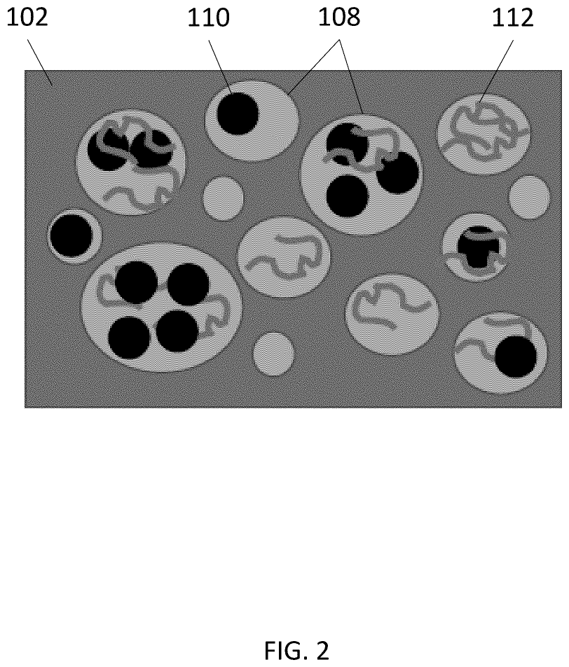

FIG. 2 depicts a magnified view of an exemplary tube body 102 comprising pores 108 loaded with a plurality of microparticles 110 in an excipient solution 112. Tube body 102 can comprise any suitable pores 108 for the loading and delivery of a drug. In some embodiments, pores 108 of tube body 102 can be described in terms of pore radius, wherein pores 108 comprise a pore radius between about 10 μm and 200 μm or greater. In some embodiments, tube body 102 can be described as having an average pore radius of about 10 μm, 20 μm, 30 μm, 40 μm, 50 μm, 60 μm, 70 μm, 80 μm, 90 μm, 100 μm, 200 μm, or greater. In some embodiments, pores 108 can be described in terms of throat radius, which is understood by persons having skill in the art to describe connections between each pore 108 through which microparticles and fluids may flow through. Contemplated throat radii are between about 1 μm and 100 μm or greater. In some embodiments, tube body 102 can be described as having an average throat radius of about 10 μm, 20 μm, 30 μm, 40 μm, 50 μm, 60 μm, 70 μm, 80 μm, 90 μm, 100 μm, or greater. In some embodiments, tube body 102 can be described in terms of porosity, which is understood by persons having skill in the art to describe a ratio or percentage of void space in a material by volume. In some embodiments, tube body 102 can have a porosity between about 10% and 50% or greater. In some embodiments, tube body 102 can have a porosity of about 10%, 20%, 30%, 40%, 50%, or greater.

Microparticles are generally understood by persons having skill in the art to refers to small particles which behave as a whole unit in terms of their transport and properties, and which typically exhibit an average particle size diameter (determined, for example, by a microscopy, electrozone sensing, or laser diffraction technique) in the range of about 0.1 to 10 μm or greater. Terms that may be used synonymously with microparticle include but are not limited to: nanoparticle, micro- and nanobubble, micelle, micro- and nanosphere, micro- and nanocapsule, micro- and nanobead, micro- and nanosome, and the like. Microparticles may comprise any structure suitable for the delivery of a desired therapeutic. For example, a microparticle may comprise a vesicle-like structure composed of a fluid core encased in a membrane comprising a lipid bilayer. Alternatively, a microparticle may comprise a hydrophilic shell and a hydrophobic core. A microparticle may also comprise one or more solid cores, or a distribution of solid or fluid deposits within a matrix.

The microparticles may be uncoated or coated to impart a charge or to alter lipophilicity. Microparticles may have a uniform shape, such as a sphere. Microparticles may also be irregular, crystalline, semi-crystalline, or amorphous. A single type of microparticle may be used, or mixtures of different types of microparticles may be used. If a mixture of microparticles is used they may be homogeneously or non-homogeneously distributed. In various aspects, the microparticle is biodegradable or non-biodegradable, or in a plurality of microparticles, combinations of biodegradable and non-biodegradable cores are contemplated.

In some embodiments, the microparticles comprise a polymer. Non-limiting examples of suitable polymers include but are not limited to PLGA, PLA, PGA, PCL, PLL, cellulose, poly(ethylene-co-vinyl acetate), polystyrene, polypropylene, dendrimer-based polymers, polyethylene glycol (PEG), branched PEG, polysialic acid (PSA), carbohydrate, polysaccharides, pullulane, chitosan, hyaluronic acid, chondroitin sulfate, dermatan sulfate, starch, dextran, carboxymethyl-dextran, polyalkylene oxide (PAO), polyalkylene glycol (PAG), polypropylene glycol (PPG), polyoxazoline, polysebacates, poly(glycerolsebacates), poly acryloylmorpholine, polyvinyl alcohol (PVA), polycarboxylate, polyvinylpyrrolidone, polyphosphazene, polyoxazoline, polyethylene-co-maleic acid anhydride, polystyrene-co-maleic acid anhydride, poly(1-hydroxymethylethylene hydroxymethylformal) (PHF), 2-methacryloyloxy-2′-ethyltrimethylammoniumphosphate (MPC), polyethylene glycol propionaldehyde, copolymers of ethylene glycol/propylene glycol, monomethoxy-polyethylene glycol, carboxymethylcellulose, polyacetals, poly-1,3-dioxolane, poly-1,3,6-trioxane, ethylene/maleic anhydride copolymer, poly (β-amino acids) (either homopolymers or random copolymers), poly(n-vinyl pyrrolidone)polyethylene glycol, propropylene glycol homopolymers (PPG) and other polyakylene oxides, polypropylene oxide/ethylene oxide copolymers, polyoxyethylated polyols (POG) (e.g., glycerol) and other polyoxyethylated polyols, polyoxyethylated sorbitol, or polyoxyethylated glucose, colonic acids or other carbohydrate polymers, Ficoll or dextran and combinations or mixtures thereof. For example, in some embodiments, the PLGA comprises any PLGA known in the art, including, but not limited to, 99:1 PLGA, 95:5 PLGA, 90:10 PLGA, 85:15 PLGA, 80:20 PLGA, 75:25 PLGA, 70:30 PLGA, 65:35 PLGA, 60:40 PLGA, 55:45 PLGA, 50:50 PLGA, 45:55 PLGA, 40:60 PLGA, 35:65 PLGA, 30:70 PLGA, 25:75 PLGA, 20:80 PLGA, 15:85 PLGA, 10:90 PLGA, 5:95 PLGA, and/or 1:99 PLGA.

The microparticles can contain or encapsulate one or more drugs or therapeutics. Contemplated drugs or therapeutics include but are not limited to local anesthetics such as: articaine, benzocaine, benzonatate, bupivacaine, chloroprocaine, cinchocaine, diclofenac-diethylamine, dimethocaine, eucaine, etidocaine, exparel, hexylcaine, levobupivacaine, lidocaine, mepivacaine, meprylcaine, oxybuprocaine, phenacaine, piperocaine, pramocaine, prilocaine, procaine, proparacaine, quinisocaine, ropivacaine, tetracaine, or trimecaine used in their native, nanoparticle, microsphere, cyclodextrin, or liposomal forms. Drugs and therapeutics can be used alone or with adjuvants to increase efficacy, which can include (either singly or in combination) epinephrine, narcotics, steroids, clonidine, dexmedetomidine, parecoxib, lornoxicam, midazolam, ketamine, magnesium sulfate, neostigmine, tonicaine, n-butyl tetracaine, adenosine, dextran, neuromuscular blocker, or butamben.

In various embodiments, the drugs or therapeutics include but are not limited to anti-viral agents, anti-bacterial agents, anti-thrombolytics, anti-fibrinolytics, chemotherapeutics, anti-hypertensives, immunotherapeutics, antibiotics, anti-inflammatory agents, pro-inflammatory agents (such as for pleurodesis), anticoagulants, procoagulants, clotting agents, anticlotting agents, antiseptics, anesthetics, analgesics, pharmaceutical agents, antibodies, small molecules, peptides, nucleic acids, and the like.

In certain embodiments, the drugs or therapeutics comprise at least one antibacterial agent. In one embodiment, the antibacterial agent is a broad-spectrum antibacterial agent. Suitable antibacterial agents include, but are not limited to, chlorhexidine and derivatives thereof, members of the bisbiguanide class of inhibitors, povidone iodine, hydrogen peroxide, doxycycline, minocycline, clindamycin, doxycycline, metronidazole, essential oil extracts (menthol, thymol, eucalyptol, methyl salicylate, metal salts (zinc, copper, stannous ions), phenols (triclosan), all quaternary ammonium compounds (cetylpyridinium chloride), surfactants (sodium lauryl sulphate, delmopinol), all natural molecules (phenols, phenolic acids, quinones, alkaloids, lectins, peptides, polypeptides, indole derivatives, flustramine derivatives, carolacton, halogenated furanones, oroidin analogues, agelasine, ageloxime D).

In some embodiments, the drugs or therapeutics are formulated using one or more pharmaceutically acceptable excipients or carriers. In one embodiment, the drugs or therapeutics comprise a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In certain cases, it may be advantageous to include isotonic agents, for example, sugars, sodium chloride, or polyalcohols such as mannitol and sorbitol.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known in the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

In certain embodiments, excipients may be selected for compatibility with a material of the underlying device. For example, excipients such as cyclohexane, methyl ethyl ketone, acetone, and dimethylformamide may be suitable excipients to pair with a device comprising UHMWPE in that UHMWPE is insoluble in the presence of the listed excipients, while an excipient comprising tetrahydrofuran may be used with brief exposure to a device comprising UHMWPE in that UHMWPE is slightly soluble in the presence of tetrahydrofuran.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials.

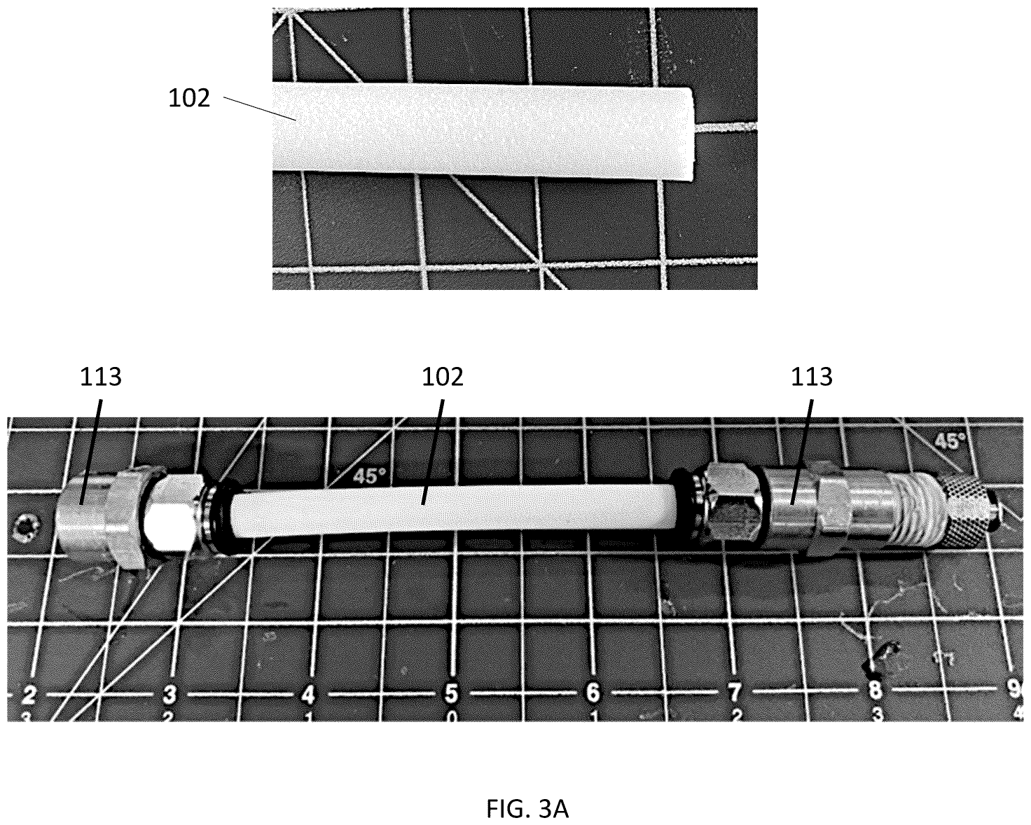

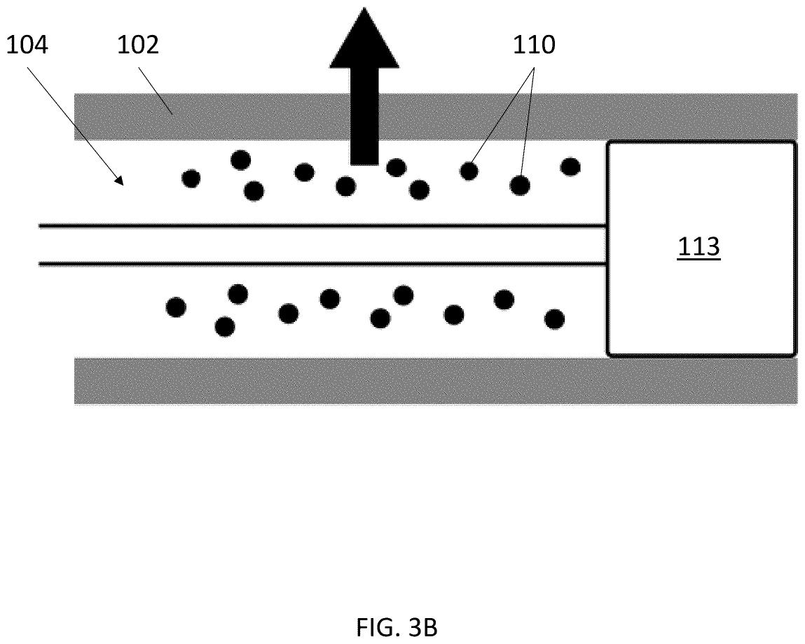

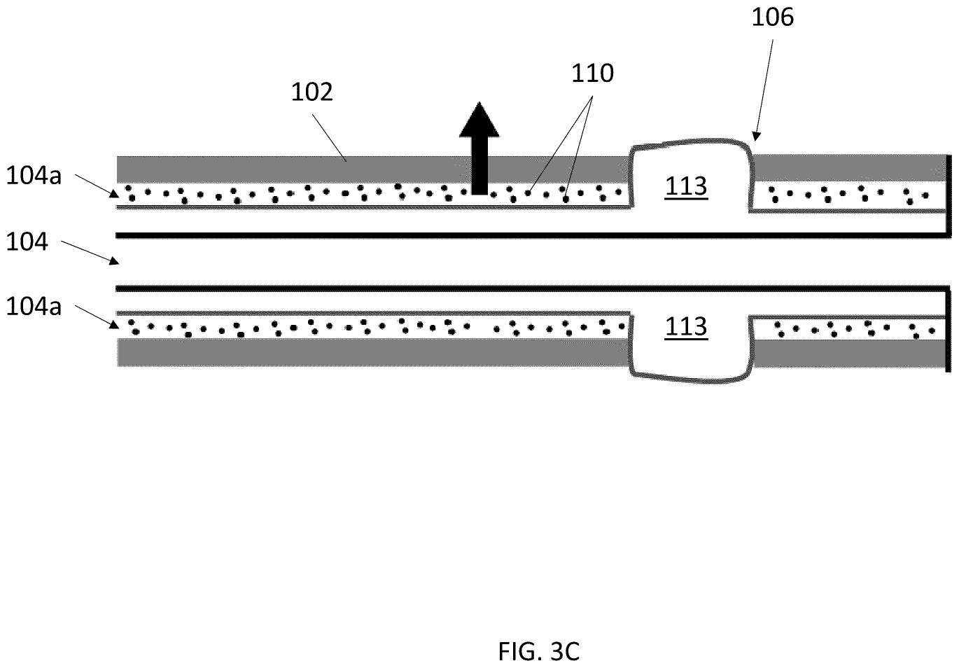

The microparticle formulations can be loaded into tube body 102 using any suitable method. For example, FIG. 3A depicts an exemplary section of tube body 102 having an occluding member 113 positioned at a proximal end and a distal end. Occluding member 113 is configured to temporarily seal lumen 104 and apertures 106 such that a microparticle formulation may be inserted or injected into lumen 104 for loading into tube body 102. Contemplated occluding members 113 include but are not limited to: liquid tight end fittings, plugs, vises, clamps, balloons, and the like. In some embodiments, tube body 102 comprises one or more lumens in addition to lumen 104, wherein the one or more lumens may receive a microparticle formulation for loading. The one or more additional lumens may be provided as a partition of lumen 104, or may be incorporated within a tube wall of tube body 102. In some embodiments, a microparticle formulation is loaded into tube body 102 under pressure, such that the microparticle formulation is pushed through tube body 102 leaving microparticles embedded within pores 108, much like a filter. In some embodiments, a tube body 102 can be immersed and incubated in a microparticle formulation. Any of the various loading steps can be followed by a drying step, whereby device 100 may be ready for use in a subject.

In some embodiments, tube body 102 can be reloaded with a microparticle formulation in situ. For example, FIG. 3B schematically depicts a cross-section of tube body 102 positioned in situ. An occluding member 113 (such as an inflatable balloon at the distal end of an inflation lumen) is introduced into lumen 104 and positioned to block a distal opening. While not pictured, the same occluding member 113 or additional occluding members 113 may be positioned to further block apertures 106. A formulation comprising microparticles 110 is flowed into the occluded lumen 104, where microparticles 110 are captured by tube body 102 in the direction of the solid black arrow by the application of a positive pressure into lumen 104. FIG. 3C schematically depicts a cross-section of another tube body 102 positioned in situ. Tube body 102 comprises a main lumen 104 and one or more additional lumens 104a positioned within tube body 102. Occluding members 113 are built into tube body 102 and are depicted in FIG. 3C as inflatable balloons fluidly connected to an inflation lumen adjacent to the one or more additional lumens 104a. Upon inflation, occluding members 113 can occlude a distal end of the one or more additional lumens 104a, and can also occlude adjacent apertures 106 if present. Similar to the schematic inn FIG. 3B, a formulation comprising microparticles 110 can be flowed into the one or more occluded additional lumens 104a, where microparticles 110 are captured by tube body 102 in the direction of the solid black arrow by the application of a positive pressure into the one or more additional lumens 104a. It should be understood that a microparticle formulation used for reloading in situ can be selected for biocompatibility, such as a microparticle suspension in saline, Lactated Ringers, intravenous sugar solution, and the like.

### Solid Cast Device

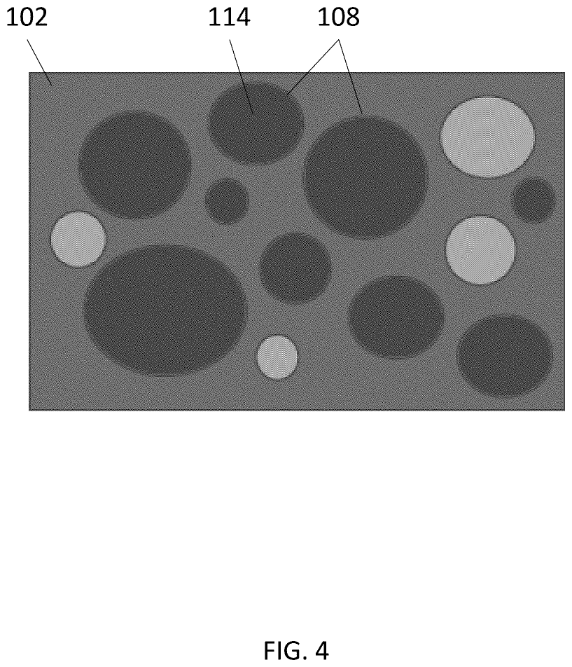

Device 100 can be loaded with drugs and therapeutics through means not limited to microparticle loading in pores. For example, in some embodiments, tube body 102 can be “solid cast” or loaded directly with nonencapsulated drug or therapeutic delivered in a solution. FIG. 4 depicts a magnified view of an exemplary tube body 102 comprising pores 108 loaded with drug or therapeutic 114. Similar to microparticle loading, tube body 104 can be contacted with a solution comprising one or more drugs or therapeutics 114, such as by immersion, flow through lumen 104, and the like. In certain embodiments, tube body 104 is saturated with the one or more drugs or therapeutics 114, such that the one or more drugs or therapeutics 114 infiltrates pores 108. In some embodiments, device 100 can be treated with a drying step. In some embodiments, device 100 can be treated with a solvent removal step. Following the drying step or the solvent removal step, drug or therapeutic 114 that has infiltrated pores 108 remain within device 100, such as in desiccated or lyophilized solution following the drying step, or embedded in drug-releasing polymer following the solvent removal step. Contemplated drugs or therapeutics 114 are described elsewhere herein. Contemplated solutions include and are not limited to polymers, solvents, and excipients described elsewhere herein.

### Surface Treated Device

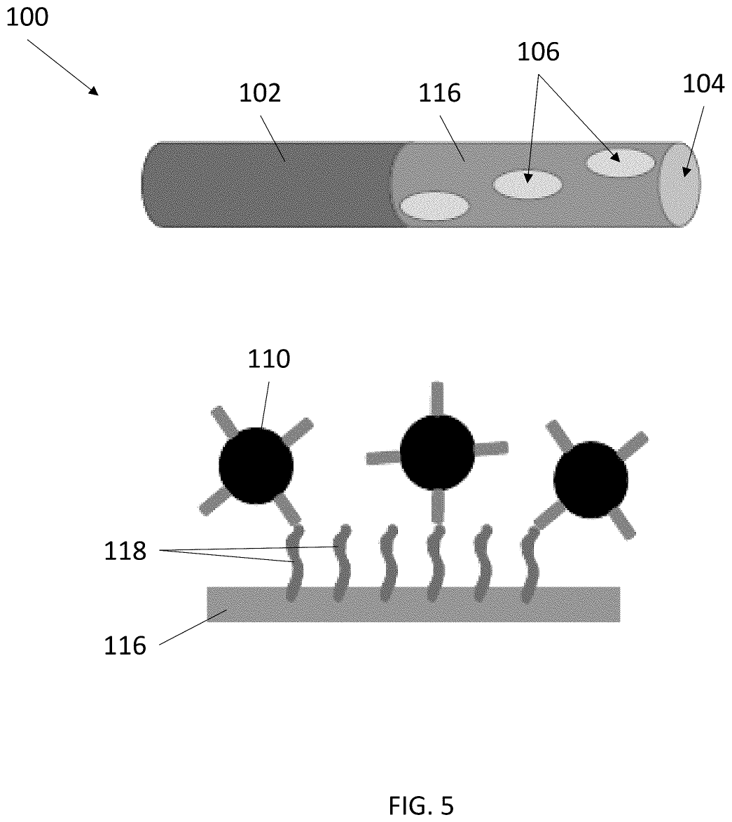

In some embodiments, tube body 102 can comprise a surface treatment 116 having a drug or therapeutic bound to the surface treatment 116 by covalent bonds 118. For example, FIG. 5 schematically depicts microparticles 110 covalently bound to surface treatment 116. In some embodiments, the surface treatment 116 is applied using an amination process, wherein a substitution reaction substitutes an ethylene diamine (EDA) molecule for a molecule on a surface of tube body 102, such as a chlorine group in a polyvinyl chloride (PVC) tube body, to leave a free amine on the tube surface. Following the amination step, the surface treatment 116 may be modified using a crosslinker (such as glutaraldehyde) and coated with a desired drug or therapeutic.

### Gel-Coated Device

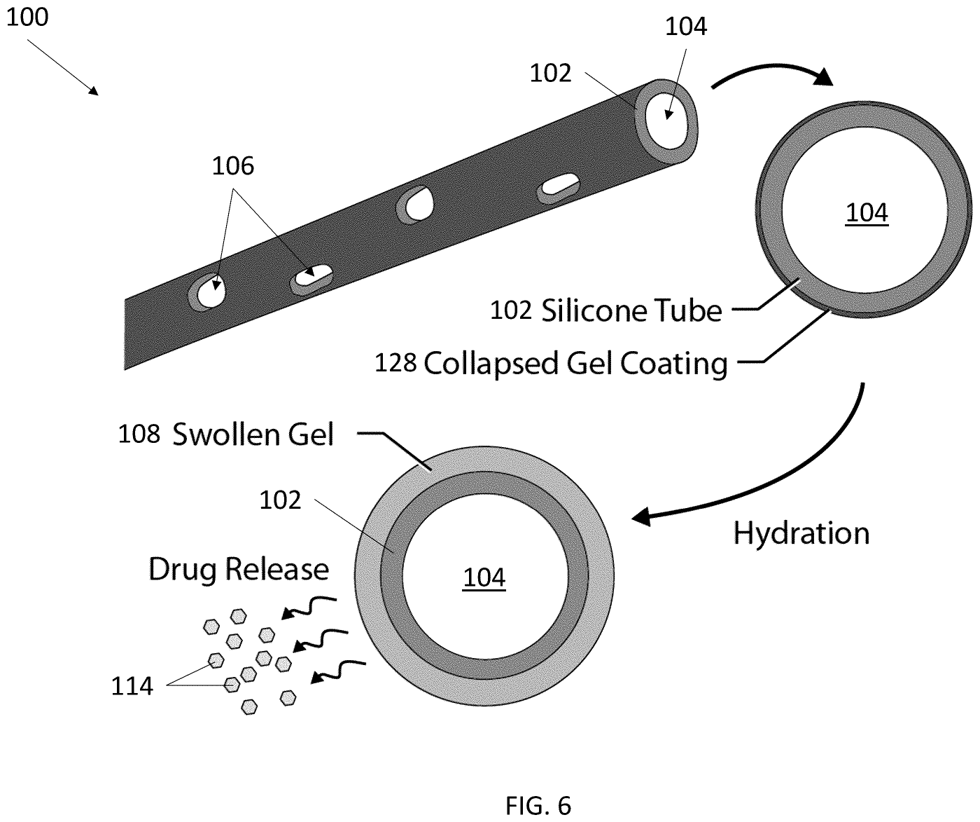

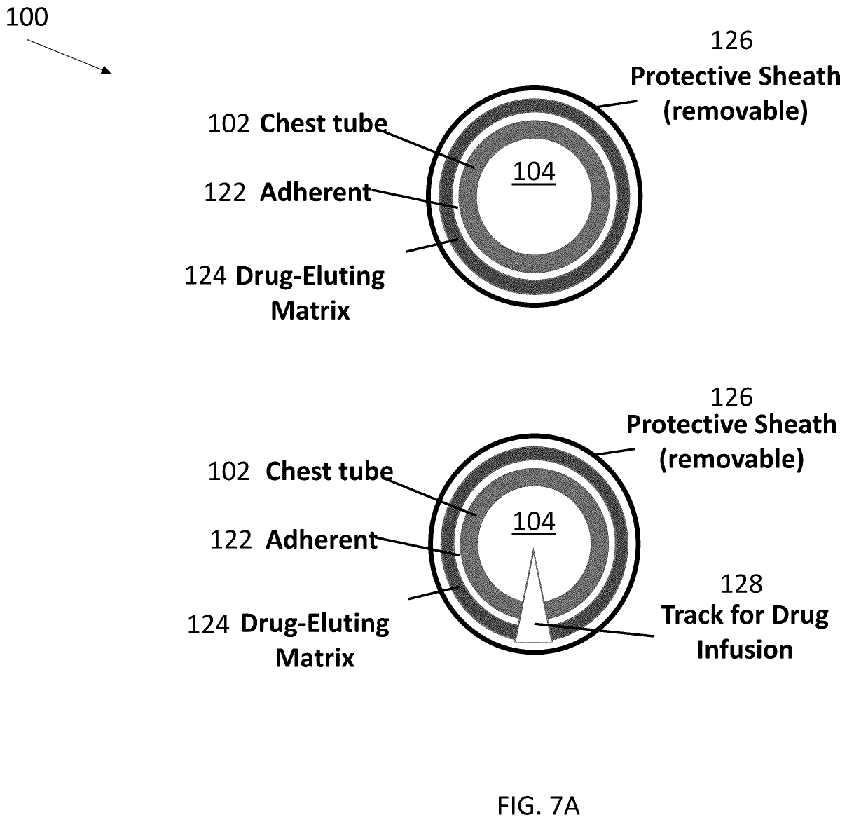

Referring now to FIG. 6, device 100 can include at least one coating positioned on the exterior or interior of tube body 102. The at least one coating can cover the entirety of tube body 102 or at least a portion of tube body 102. In some embodiments, the at least one coating is lubricating, such that device 100 is slippery to the touch, can be inserted into a subject's anatomy with minimal force, and rests within the subject's anatomy with minimal friction. In some embodiments, the at least one coating is drug-eluting, such that drugs or therapeutics embedded within the at least one coating are deliverable to a target site. The drugs or therapeutics may be delivered by diffusing or leaching out of the at least one coating. The drugs or therapeutics may also be delivered by degradation or dissolution of the at least one coating. In various embodiments, the at least one coating may be layered, wherein each layer comprises a different drug or therapeutic or a different delivery rate. The at least one coating can be constructed from any suitable material, including but not limited to an alginate, hydrogel, agarose/modified agarose, collagen I, polycaprolactone, polyurethanes, polyester, polyethylene, or the like.

In the embodiment depicted in FIG. 6, the at least one coating on device 100 comprises at least one gel coating 120 having a collapsed state and a swollen state. In the collapsed state, gel coating 120 can be collapsed or desiccated such that an amount of fluid is removed from gel coating 120. Gel coating 120 can thereby be compacted in size or thickness, hardened, dried, or combinations thereof. In the swollen state, gel coating 120 can be hydrated such that an amount of fluid is absorbed into gel coating 120. Gel coating 120 can thereby swell in size or thickness, soften, lubricate, or combinations thereof. The fluid can be any suitable fluid, such as an ex vivo solution or aqueous bath or an in vivo bodily fluid. In various embodiments, gel coating 120 can further comprise one or more drugs and/or therapeutics 114 embedded within.

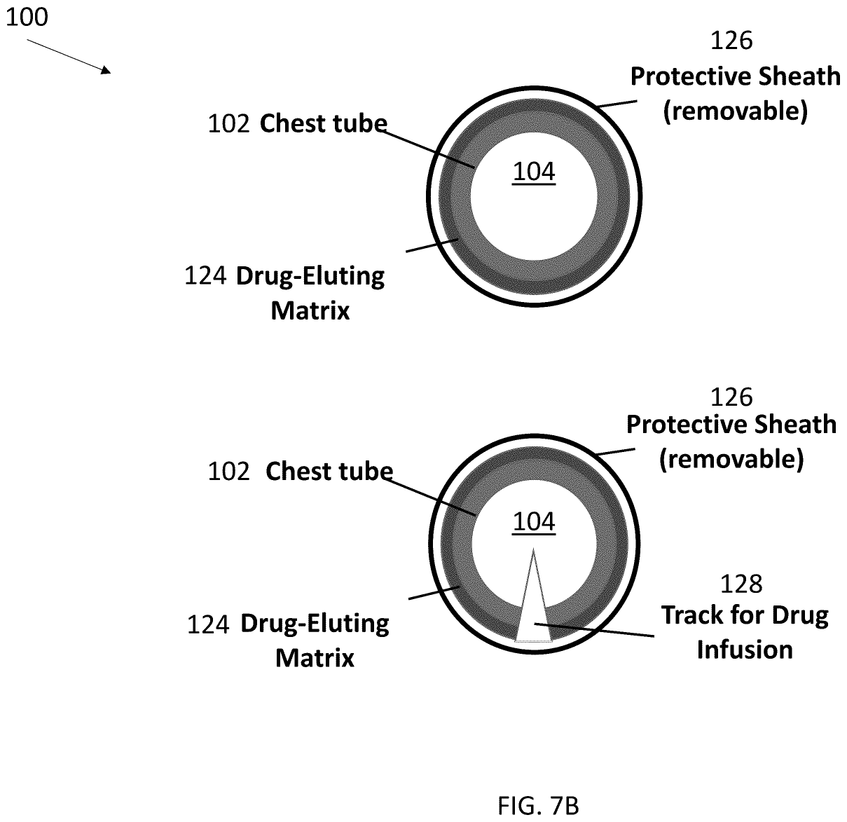

In the embodiment depicted in FIG. 7A, the at least one coating on device 100 comprises at least one drug-eluting matrix 124 adhered to tube body 102 by an adherent 122, such as a glue, a paste, a cement, or the like. In other embodiments, drug-eluting matrix 124 can be directly bonded to tube body 102. In certain embodiments, device 100 further comprises one or more reservoirs or tracks 128, wherein a drug or therapeutic can be stored for temporary delivery or channeled for continuous infusion to a target site. The reservoirs or tracks 128 can extend throughout the entire length of tube body 102 or for at least a portion of the length of tube body 102. Device 100 can further comprise a protective sheath 126 that encases at least a portion of tube body 102, or at least the portions of tube body 102 containing a drug or therapeutic. Sheath 126 can preserve the composition of drug-eluting matrix 124 and reservoir or track 128 during storage and transport and can be removed prior to or after insertion into a subject.

In the embodiment depicted in FIG. 7B, the at least one coating on device 100 comprises at least one drug-eluting matrix 124 directly adhered to tube body 102. In other embodiments, drug-eluting matrix 124 can be directly bonded to tube body 102. In certain embodiments, device 100 further comprises one or more reservoirs or tracks 128, wherein a drug or therapeutic can be stored for temporary delivery or channeled for continuous infusion to a target site. The reservoirs or tracks 128 can extend throughout the entire length of tube body 102 or for at least a portion of the length of tube body 102. Device 100 can further comprise a protective sheath 126 that encases at least a portion of tube body 102, or at least the portions of tube body 102 containing a drug or therapeutic. Sheath 126 can preserve the composition of drug-eluting matrix 124 and reservoir or track 128 during storage and transport and can be removed prior to or after insertion into a subject.

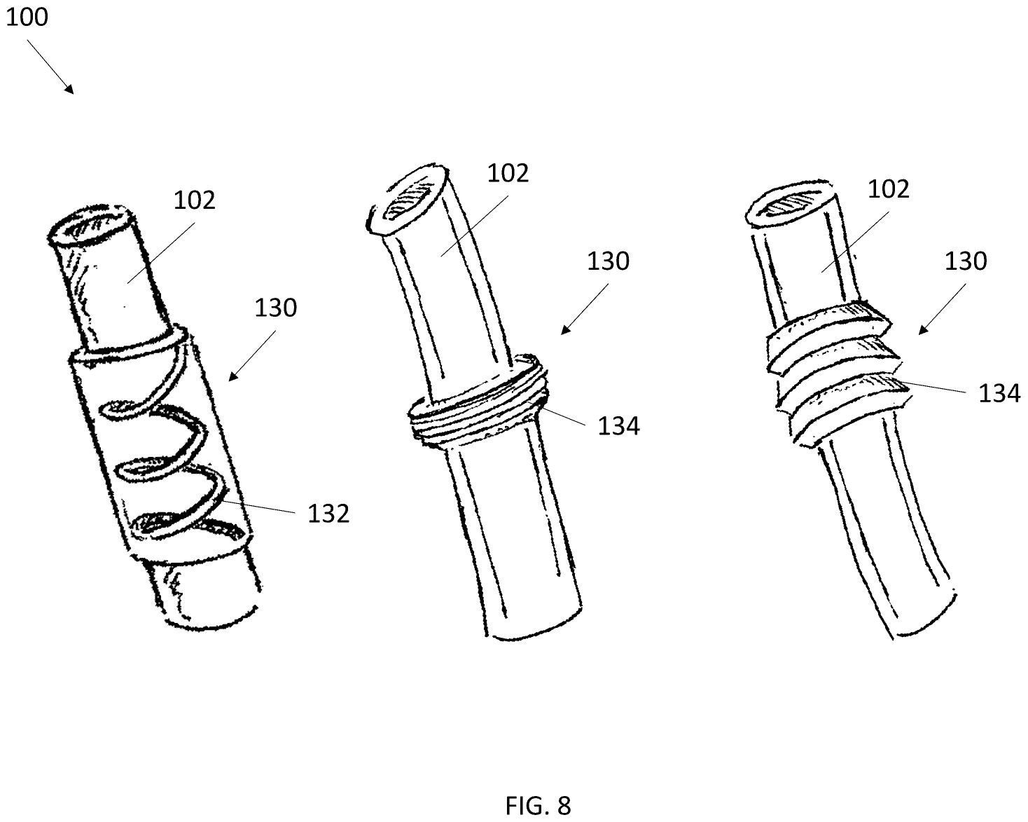

Referring now to FIG. 8, embodiments of device 100 are depicted with flexible regions 130. Device 100 can include one or more flexible regions 130, which can help devices 100 exert less pressure on a subject's anatomy by providing softer surfaces that rest against the subject's tissues and by conforming to the subject's body shape. Flexible regions 130 can also reduce or dampen mechanical discomfort by yielding to movement and applications of longitudinal force, such as acute pulling or in advertent catching of an external portion of device 100. In some embodiments, a flexible region 130 can include one or more springs 132 connecting adjacent sections of tube body 102. The one or more springs 132 can be encased in a flexible and stretchable membrane that matches the range of motion of the one or more springs 132 while preserving the continuity of the at least one lumen 104 within device 100. In some embodiments, a flexible region 130 can include one or more corrugations 134. The one or more corrugations 134 can be compacted (FIG. 8, middle) and expanded (FIG. 8, right), such as a bendable straw or an accordion.

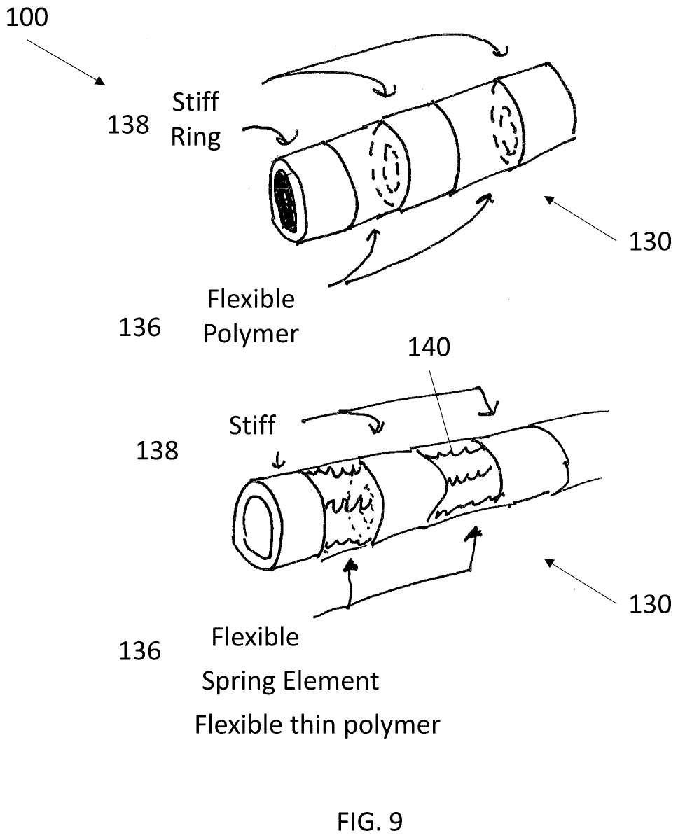

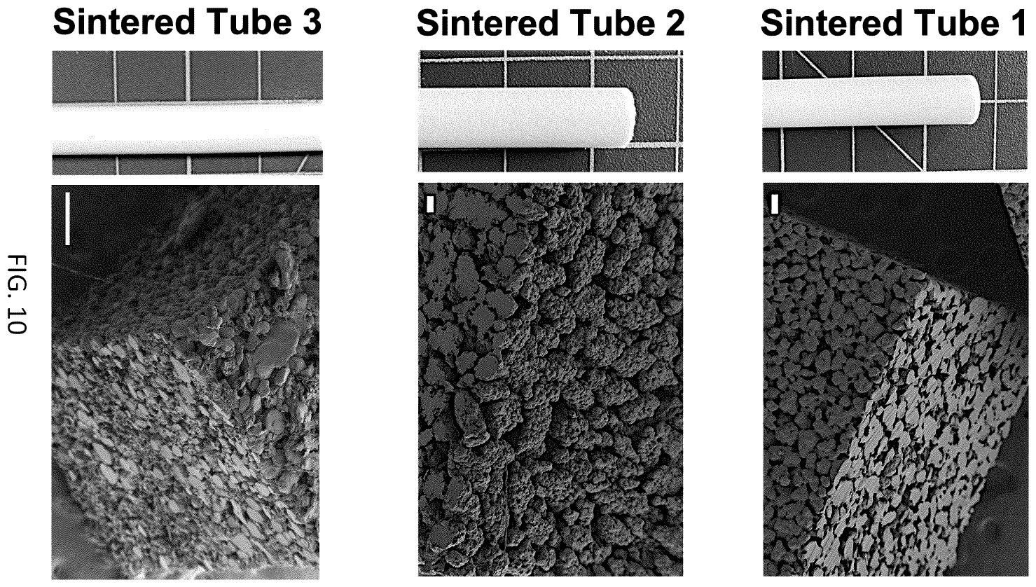

Referring now to FIG. 9, further embodiments of device 100 are depicted with flexible regions 130 constructed from alternating flexible sections 136 and stiff sections 138 of tube body 102. Flexible sections 136 afford device 100 with flexibility and pliability while stiff sections 138 maintains radial stiffness to prevent collapse of device 100, either by physical pressures or vacuum pressures. The flexible sections 136 can comprise a flexible and stretchable membrane and the stiff sections 138 can comprise a section of tube body 102 or reinforced tube body 102. In some embodiments, the flexible sections 136 can be supplemented with one or more springs 140.

In certain embodiments, the present invention relates to any tubing or implantable device that would benefit from the lubricated and drug delivering coatings and regions comprising enhanced flexibility described herein. For example, suitable tubes include but are not limited to chest tubes, endotracheal tubes, Foley catheters, surgical drains, and any other tubing that is wholly or partially placed within a subject. Other devices that are wholly or partially implanted in a subject that can include the various coatings and flexible features described herein include prosthetics, orthopedic implants, breast implants, surgical screws, needles, staples, and the like.

The devices of the present invention can be made using any suitable method known in the art. The method of making may vary depending on the materials used. For example, components of the device comprising a metal may be milled from a larger block of metal or may be cast from molten metal. Likewise, components of the device substantially comprising a plastic or polymer may be milled from a larger block or injection molded. In some embodiments, the devices may be made using 3D printing or other additive manufacturing techniques commonly used in the art. Porous structures may be fabricated using methods including but not limited to sintering, casting with porogens, thermally induced phase separation, gas foaming, freeze-drying, electrospinning, and the like.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out exemplary embodiments of the present invention, and are not to be construed as limiting in any way the remainder of the disclosure.

### Example 1: Microparticle Loading in Porous UHMWPE Tubes

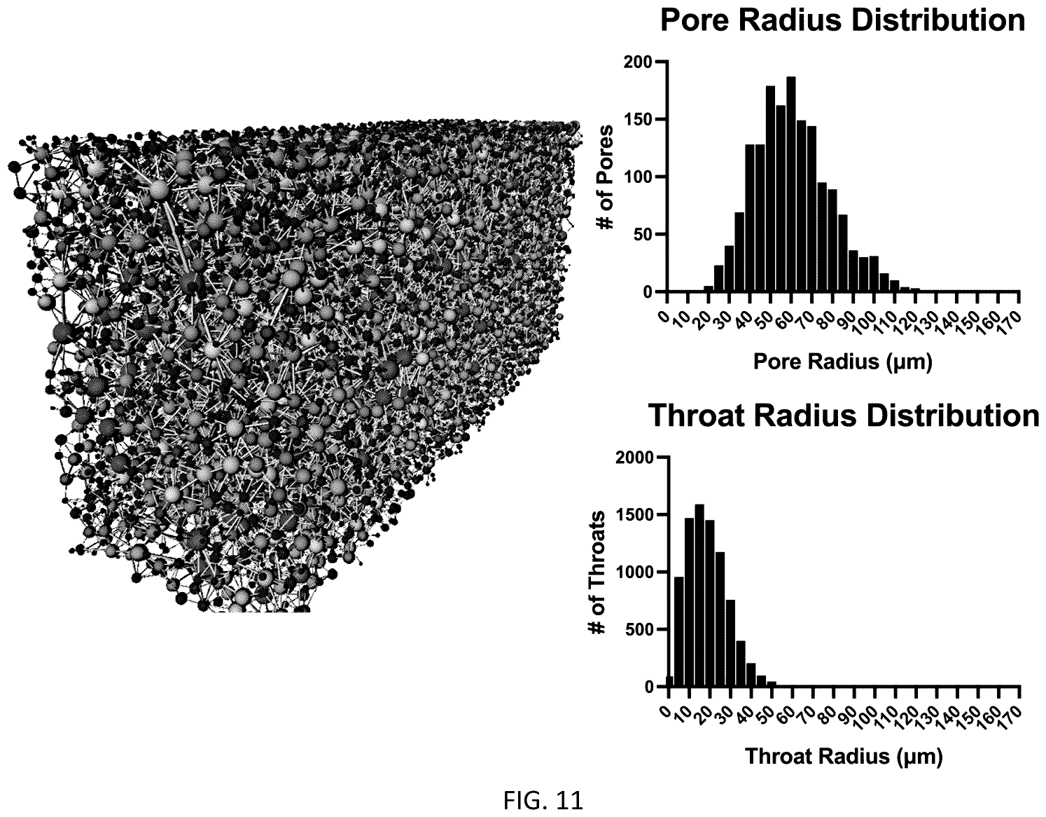

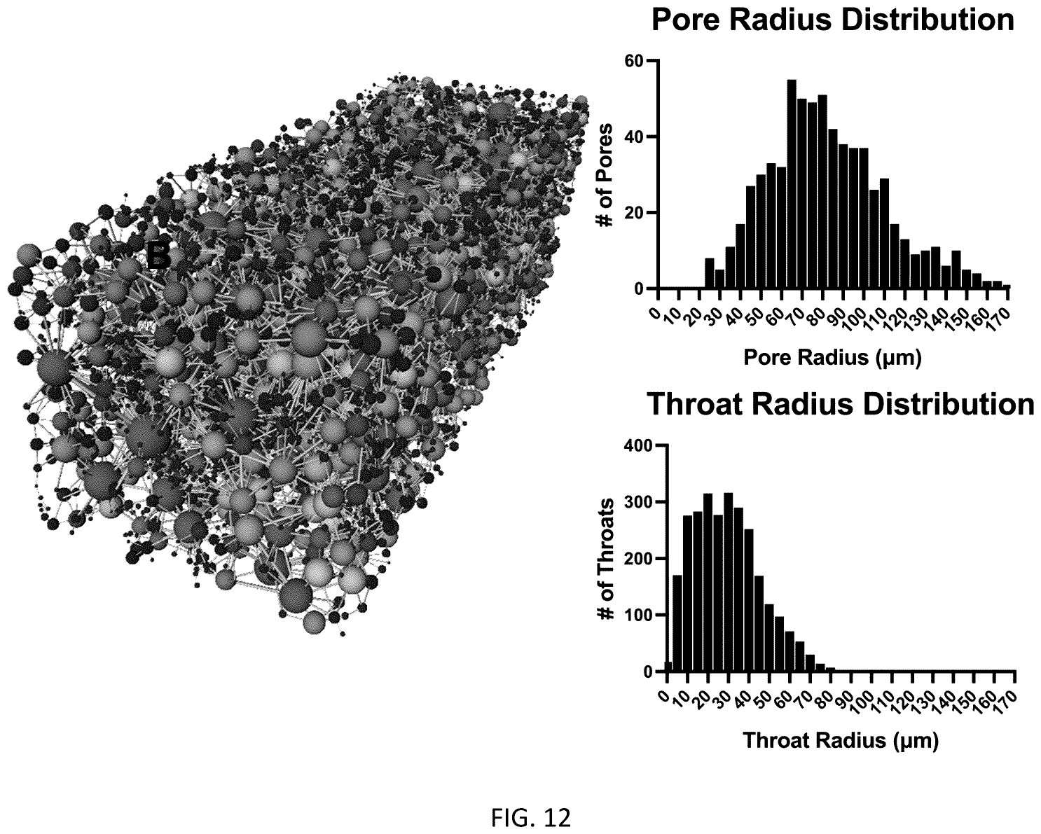

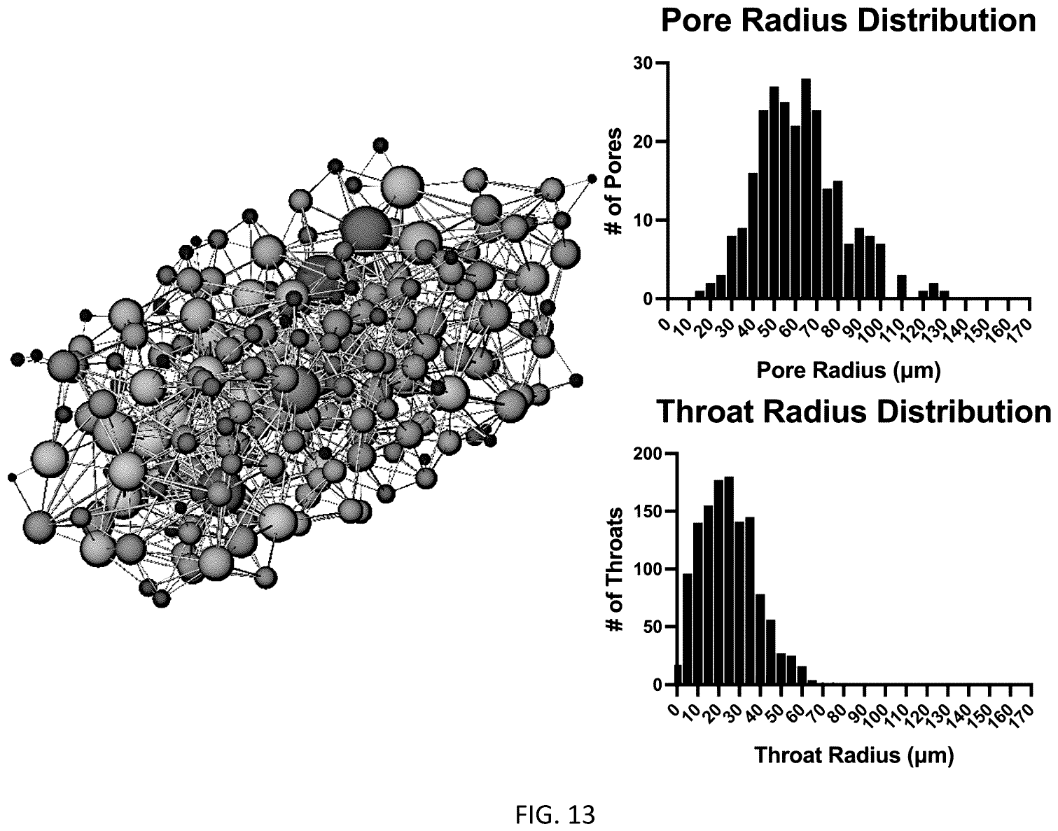

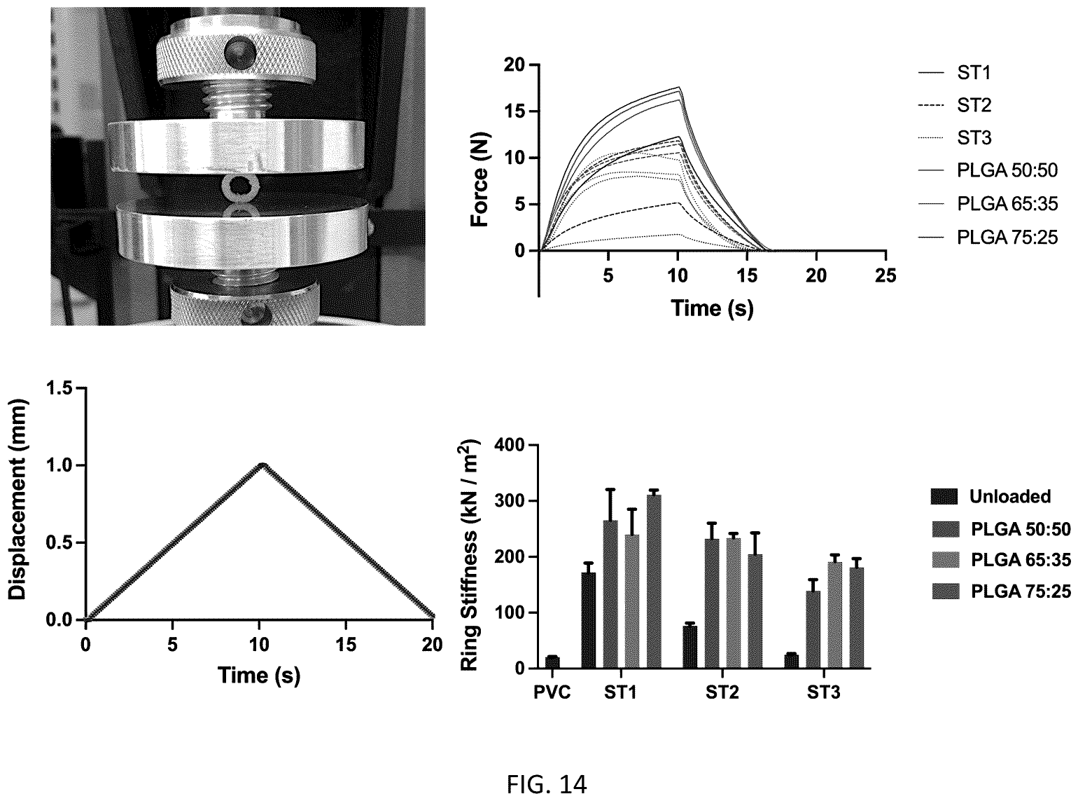

Porous UHMWPE tubes were fabricated with a 15-20 μm throat radius (hereinafter UHMWPE 15 or Sintered Tube 1 (ST1)), a 30-40 μm throat radius (hereinafter UHMWPE 30 or Sintered Tube 2 (ST2)), and Sintered Tube 3 (ST3) with about 35% to 40% porosity for each group. ST1, ST2, and ST3 were manufactured using different sintered conditions and particles (e.g., particles with different particle size) in order to prepare sintered tubes with structural differences (e.g., different pore size). SEM images shows the grain structure of the UHMWPE 15 (also referred to as ST1), UHMWPE 30 (also referred to as ST2), and ST3 (FIG. 10 right, middle, and left, respectively) achieved through a sintering fabrication process. Pore analysis of the UHMWPE 15 (also referred to as ST1) (FIG. 11) showed a porosity of about 33.6%, an average pore radius of about 60.6 μm, and an average throat radius of about 18.7 μm. Pore analysis of the UHMWPE 30 (also referred to as ST2) (FIG. 12) showed a porosity of 37.1%, an average pore radius of about 82.1 μm, and an average throat radius of about 29.6 μm. Pore analysis of the ST3 is also shown in FIG. 13 demonstrating a porosity of 42.6%, an average pore radius of about 62.1 μm, and an average throat radius of about 24.9 μm. The mechanical properties of ST1, ST2, and ST3 compared to the control PVC tube were also assessed as shown in FIG. 14. Collectively, these results indicated that ST3 was more compliant for clinical applications when compared to ST1, ST2, or PVC.

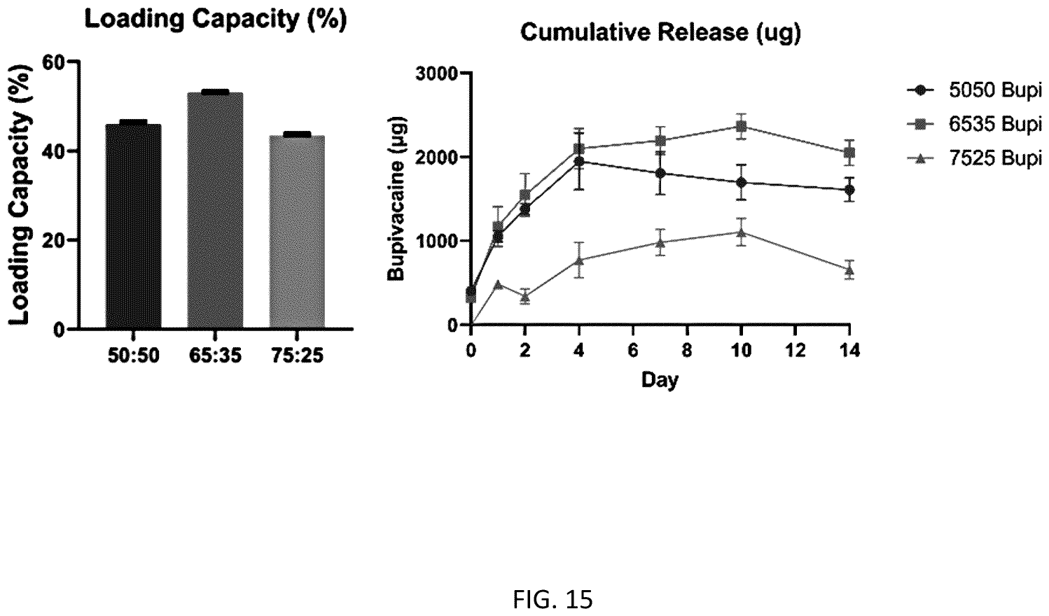

The UHMWPE 15 tubes (also referred to as ST1) were loaded with PLGA microparticles in three different formulations: 50:50 PLGA at a 50:50 ratio with bupivacaine, 65:35 PLGA at a 50:50 ratio with bupivacaine, and 75:25 PLGA at a 50:50 ratio with bupivacaine. Microparticle loading capacity analysis demonstrates that at least 40% of the total mass of the microparticles is bupivacaine (FIG. 15, left). The microparticles were mixed into a 10% (w/v) polyvinyl alcohol solution and loaded via syringe into the pores of the tubes (FIG. 3A, bottom). The tubes were then cut into 1 cm sections and immersed in PBS for drug release analysis, where each formulation demonstrated sustained release over at least a 14 day period (FIG. 15, right).

### Example 2: Solid Casting in Porous UHMWPE Tubes

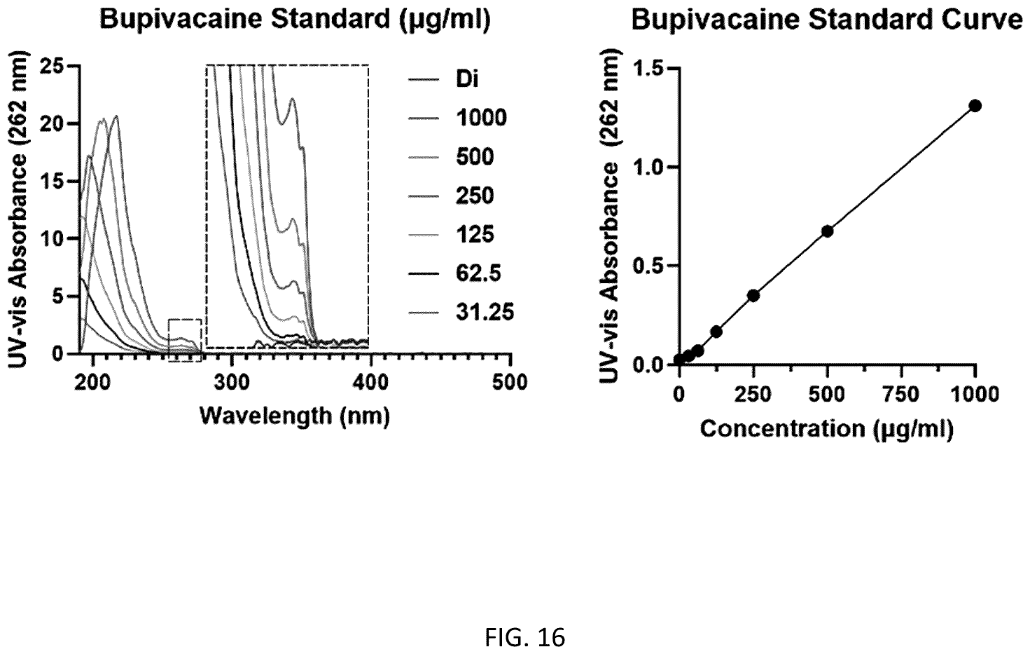

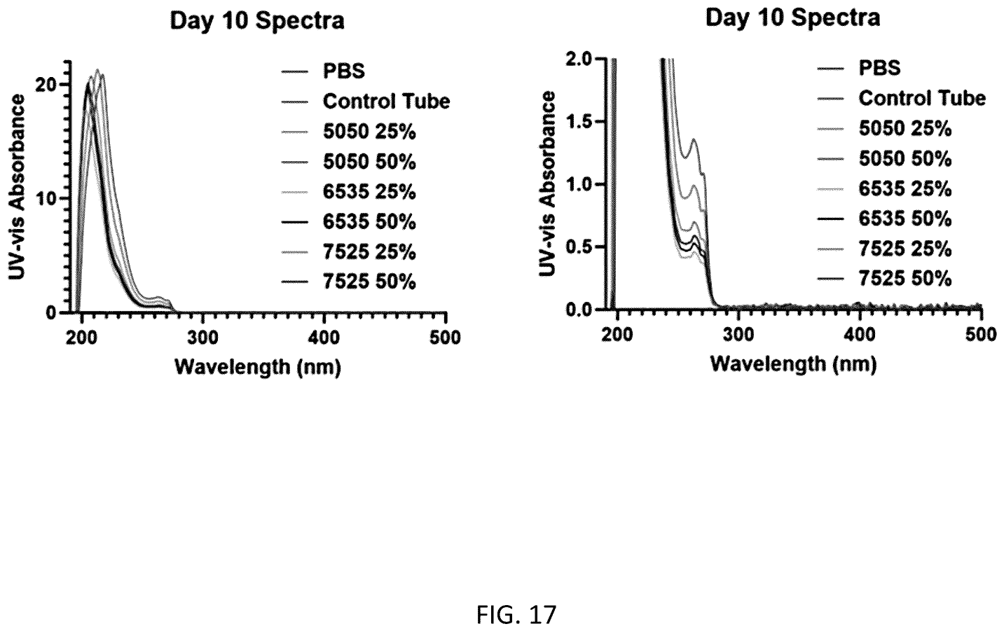

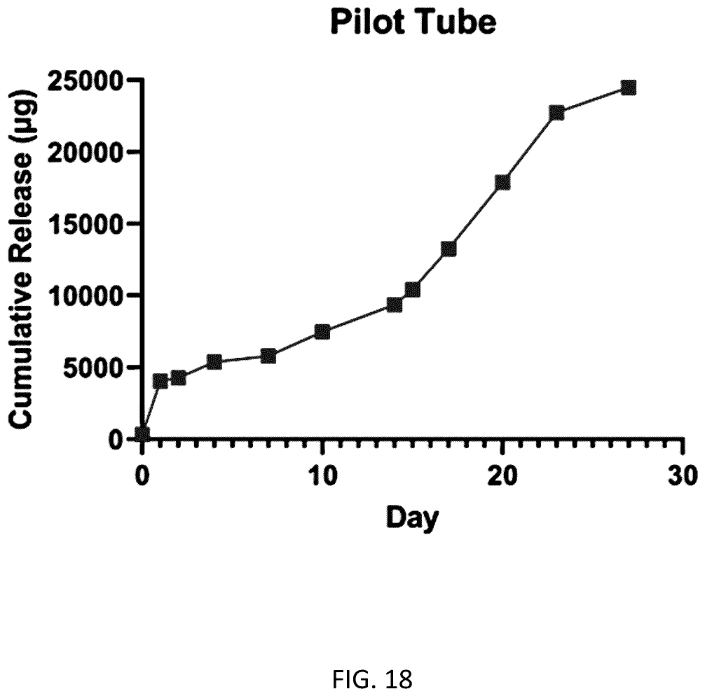

Solid casting refers to the saturation of the porous tube walls of drug or therapeutic in solution to load the tube, followed by a drying step. 6 formulations were tested through a combination of three PLGA formulations (50:50, 65:35, and 75:25) and two bupivacaine ratios (25% and 50%). Release analysis was performed by comparing the loaded weight prior to release analysis and the weight after analysis over a set length of tube to calculate percent released into solution. FIG. 16 shows the spectra of bupivacaine standards used to convert UV-vis absorbance to bupivacaine concentration. FIG. 17 shows a sampling of recorded spectra that has a similar morphology to that of pure bupivacaine, confirming that the recorded spectra is that of bupivacaine. A pilot release study was performed using a UHMWPE 15 tube (also referred to as ST1) loaded with a 65:25 PLGA, 25% bupivacaine formulation, with results showing sustained release over at least 27 days (FIG. 18).

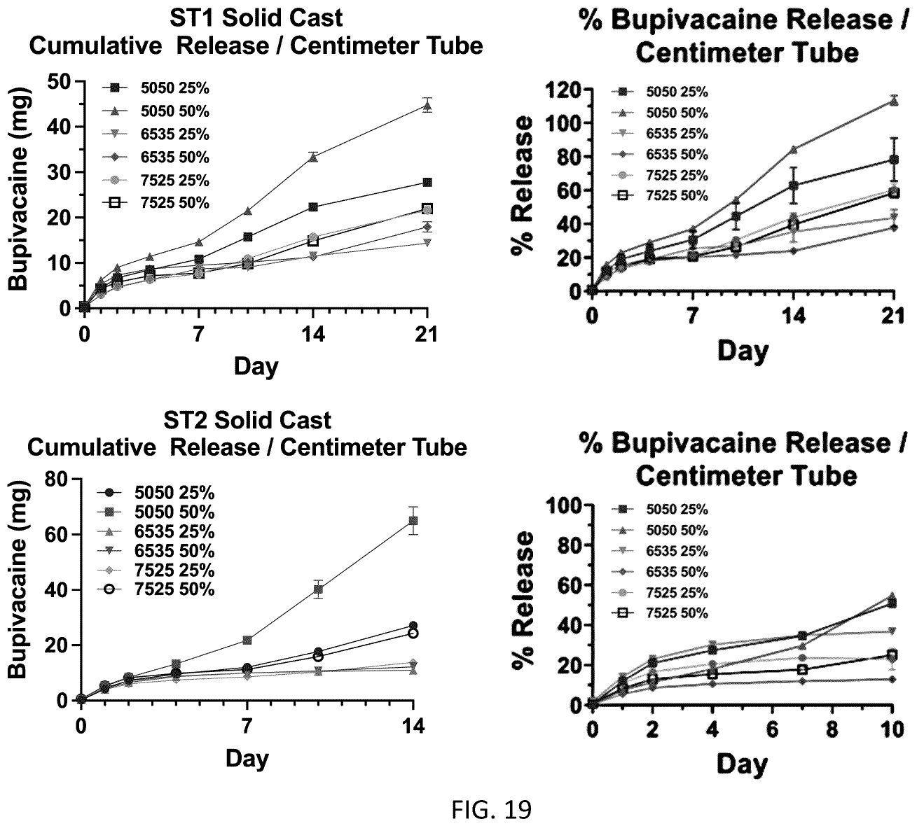

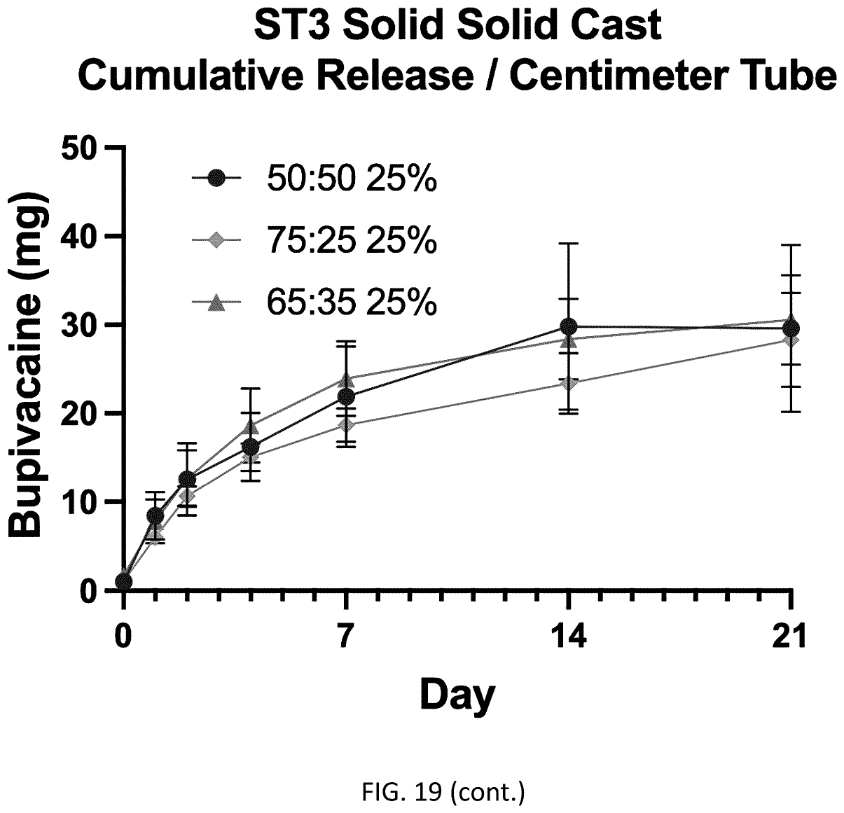

Each of the six formulations were loaded into UHMWPE 15 tube (also referred to as ST1), UHMWPE 30 tube (also referred to as ST2), and ST3 with release analysis conducted for 21, 10, and 21 days, respectively (FIG. 19). The results demonstrate that solid casting loaded catheters are feasible in delivering bupivacaine at therapeutic levels.

### Example 3: Microparticle Loading in Porous PVC Tubes

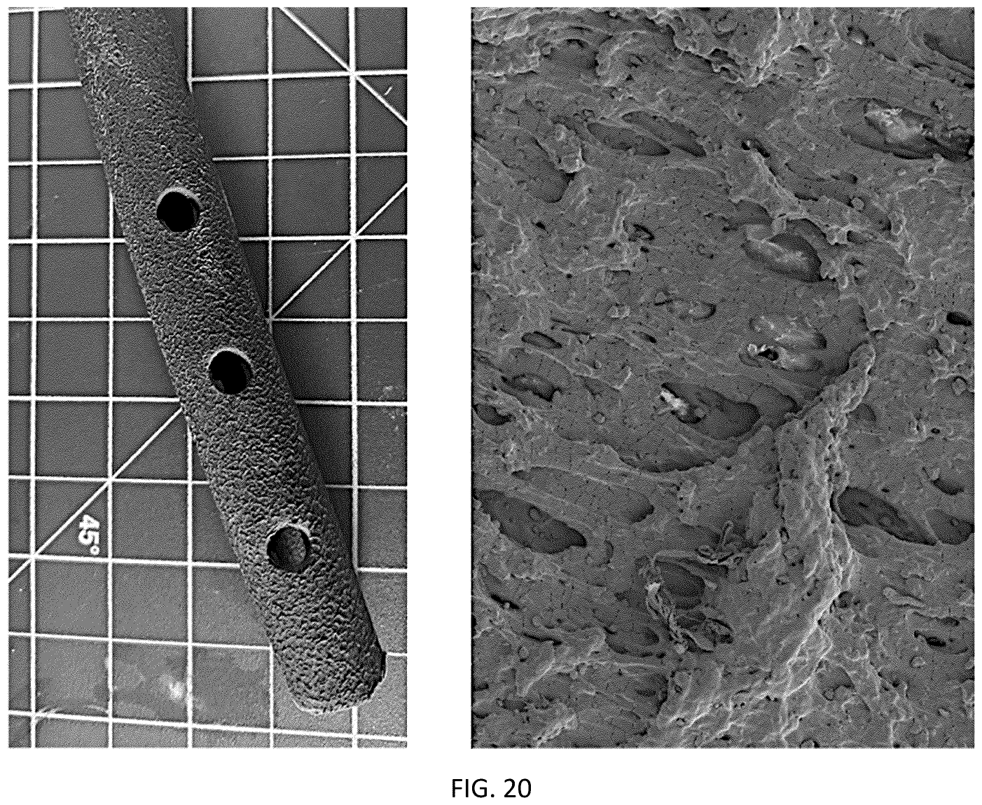

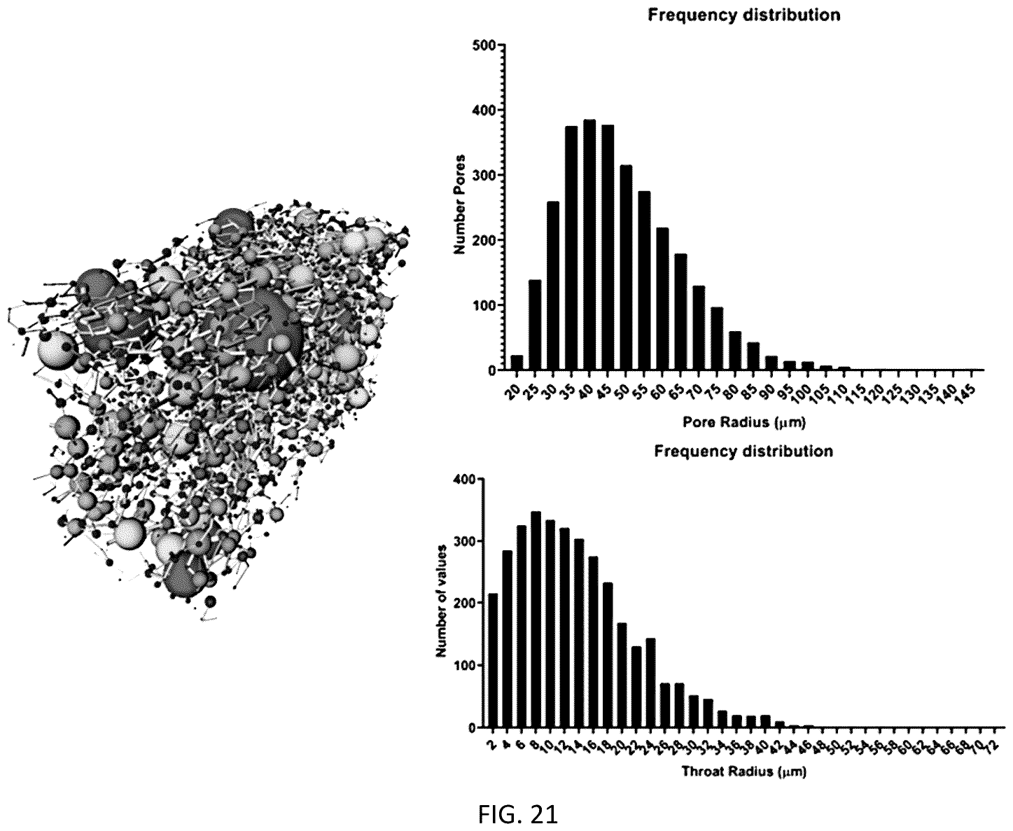

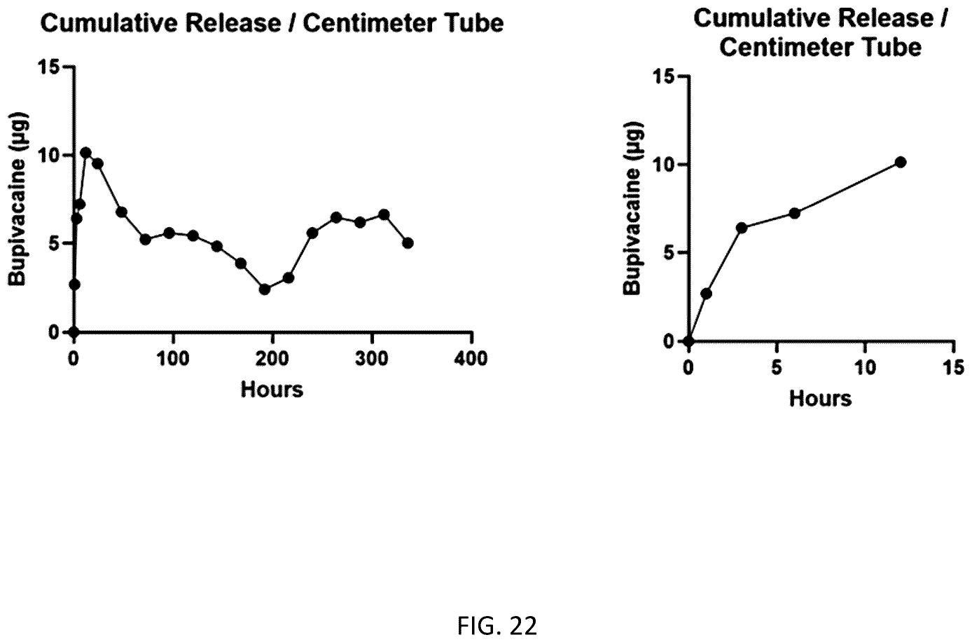

A prototype porous PVC tube is shown in FIG. 20. Pore analysis (FIG. 21) showed a porosity of 24.2%, an average pore radius of 49.0 μm, and an average throat radius of 13.8 μm. Porous PVC tube was loaded with microparticles containing 75:25 PLGA microparticles in a manner similar to Example 1. Drug release analysis shows a peak at 12 hours in this particular sample followed by a steady sustained release (FIG. 22).

### Example 4: Surface Modification in Solid PVC Tubes

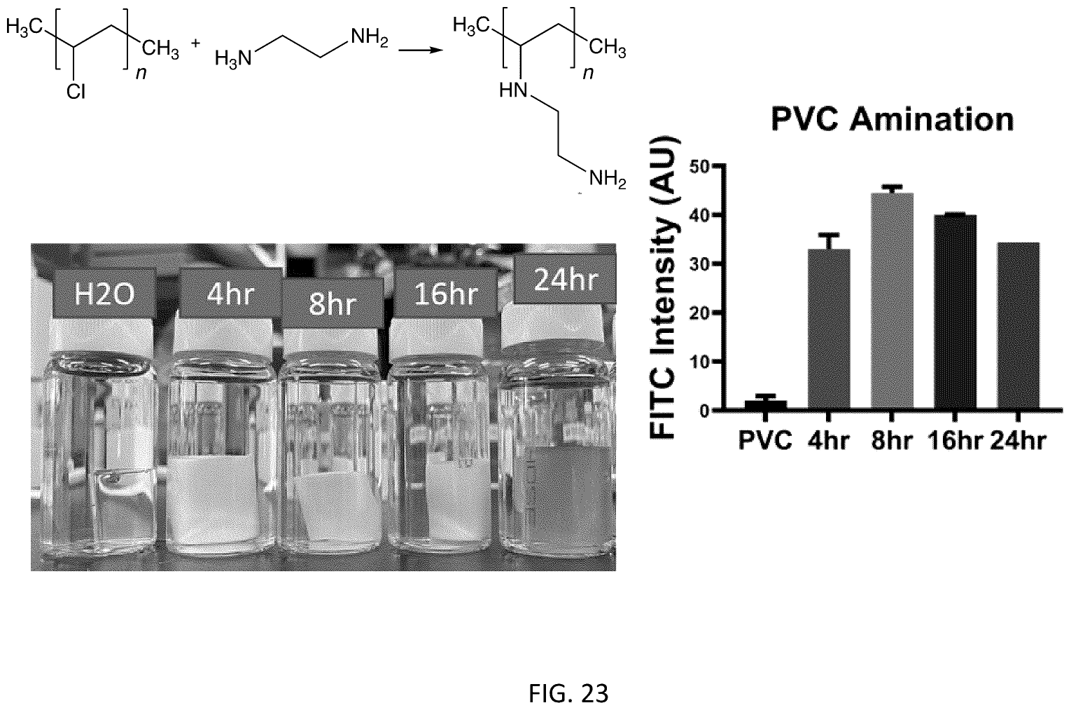

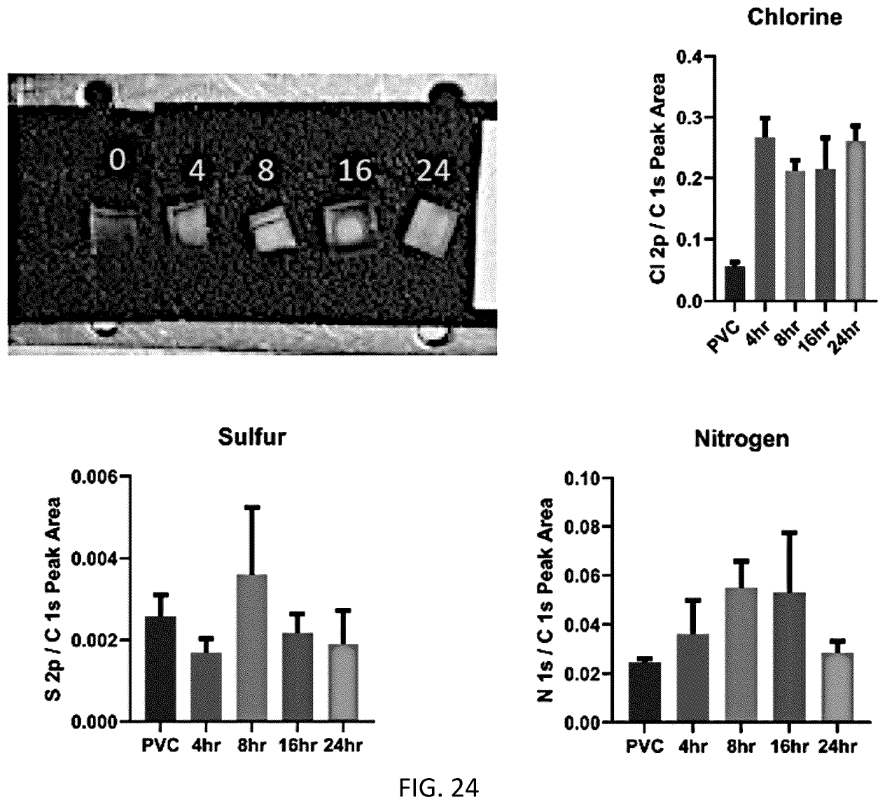

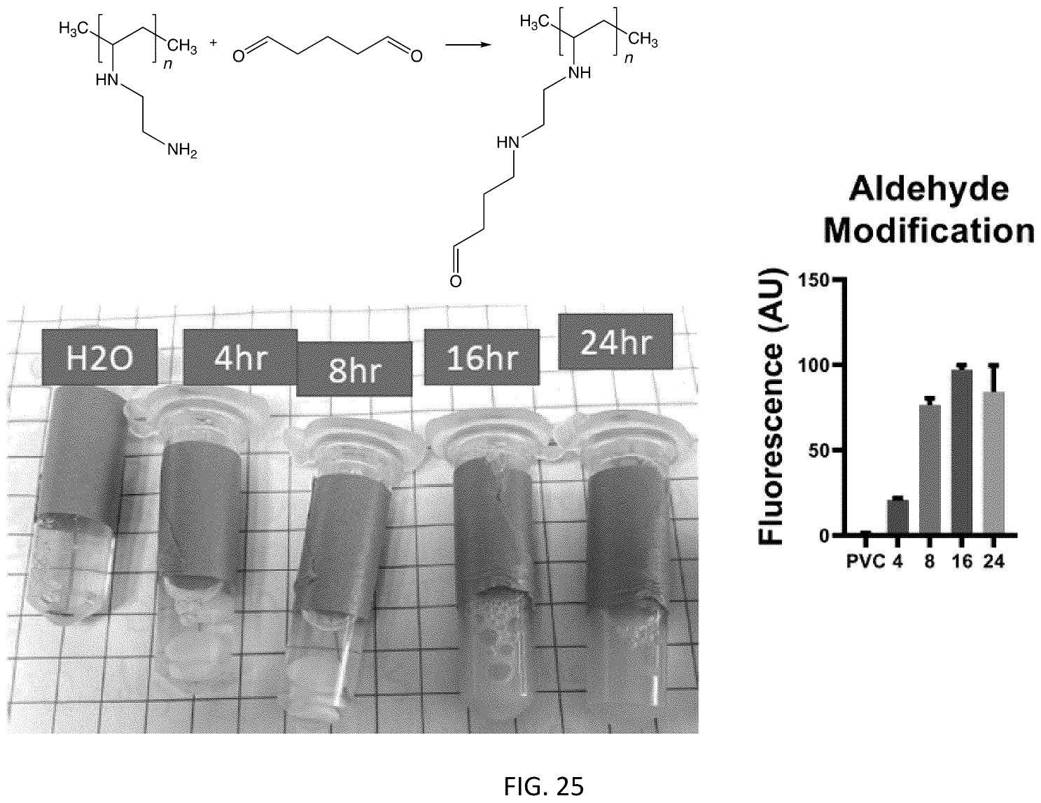

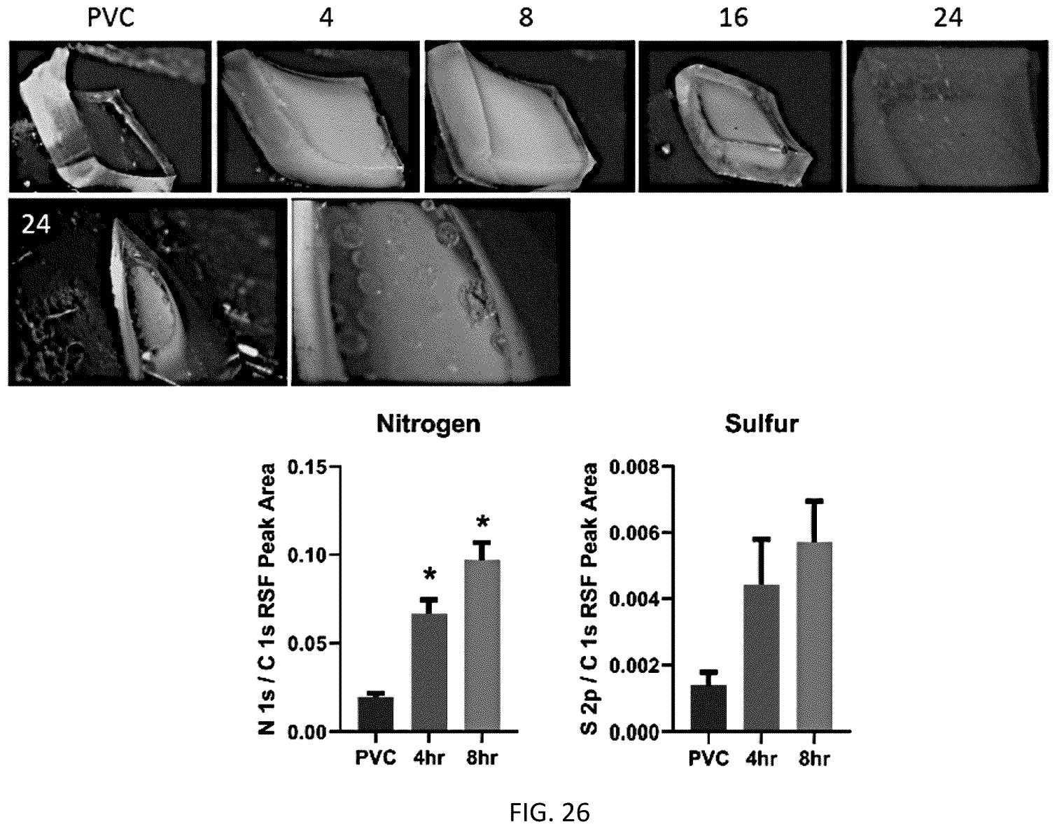

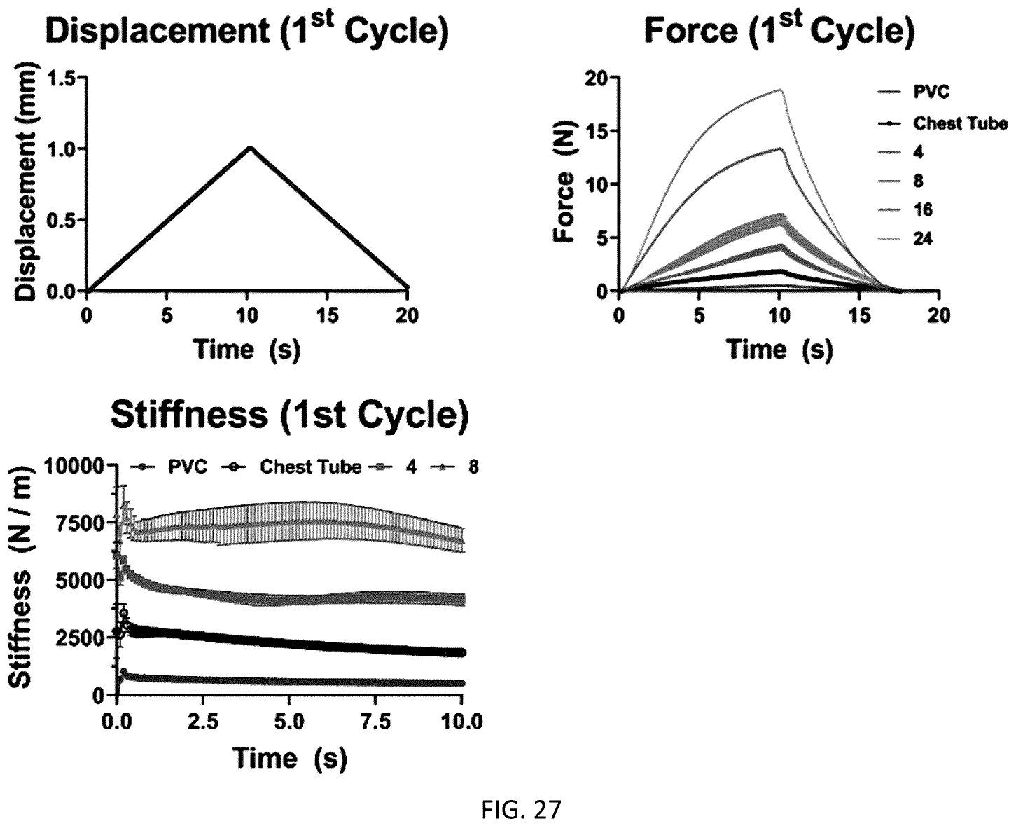

Microparticle coated PVC tubes are generated through an amination process (a substitution reaction whereby a chlorine group on the surface of the PVC tube is substituted with an EDA molecule, leaving a free amine on the surface of the tube). The process further includes a glutaraldehyde modification step and a step of coating the tube with the desired drug or therapeutic. FIG. 23 shows the amination reaction, where FITC reaction with free amines on the tube surface showed a maximum intensity, and therefore a greater number of free amines, at 8 hours incubation in EDA. Three surface moieties (nitrogen, sulfur, chlorine) were analyzed in FIG. 24. Once again, the greatest presence of surface nitrogen groups (and therefore free amines) occurs at 8 hours incubation in EDA. FIG. 25 and FIG. 26 show the results of glutaraldehyde modification of the different EDA incubation groups, where the glutaraldehyde colorimetric assay shows that the 8, 16, and 24 hour EDA incubation times had strong glutaraldehyde reactions, with the 16 hour having the greatest signal. Following glutaraldehyde modification, the tubes were reacted with a sulfur-containing molecule, with a greater sulfur content indicating more glutaraldehyde groups present on the tube surface. The results demonstrate and corroborate that the 8 hour aminated tubes have the most amine groups and the most glutaraldehyde groups. The aminated tubes were also subjected to mechanical testing (FIG. 27). Longer EDA incubation times led to stiffer tubes. For example, tubes aminated for 4 and 8 hours had a 2× and 3× increase in stiffness, respectively. These results demonstrate that an 8 hour amination period is effective in producing PVC tubes having ideal mechanical and functional properties that can be modified with any desired drug or therapeutic microparticle or molecule.

### Example 5: Silicone-Containing Devices

Furthermore, the suction through the porous tube may remove some of the drug during use in a patient. To prevent loss of suction and/or to minimize or eliminate the subsequent drug removal, a prototype comprising an inner tube that is constructed from silicone and a drug eluting outer tube was also developed and described in FIG. 28 The prototype tube has a 1 mm thick inner silicone tube that was attached through the addition of an adhesive (silicone sealant). However, in certain embodiments, the silicone tube could be much thinner (for example, 250-500 microns thick) to minimize the effect on mechanics.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A tube device comprising:
an elongate tube body having an exterior surface, an interior surface, and at least one lumen extending between a proximal end and a distal end; and
at least one drug loaded on the tube body.

2. The device of claim 1, wherein the tube body is porous, such that the at least one drug is loaded within pores of the porous tube body.

3. The device of claim 2, wherein the at least one drug is encased or embedded in a microparticle.

4. The device of claim 3, wherein the microparticle comprises a poly(lactide-co-glycolides) (PLGA).

5. The device of claim 4, wherein the PLGA is selected from the group consisting of 75:25 PLGA, 70:30 PLGA, 65:35 PLGA, 60:40 PLGA, and 50:50 PLGA.

6. The device of claim 2, wherein the at least one drug is loaded with a desiccated or lyophilized solution within the pores.

7. The device of claim 2, wherein the at least one drug is loaded in a drug-release polymer within the pores following removal of at least one solvent.

8. The device of claim 2, wherein the tube body is reloadable with the at least one drug by occluding the at least one lumen and introducing the at least one drug in a solution into the at least one lumen under a positive pressure such that the at least one drug is filtered by the porous tube body.

9. The device of claim 1, wherein the at least one drug is loaded in a drug-eluting coating positioned on the exterior surface.

10. The device of claim 1, wherein the tube body comprises at least one flexible region positioned between the proximal end and the distal end.

11. The device of claim 1, wherein the tube body has a length between about 10 cm to about 100 cm.

12. The device of claim 1, wherein the tube body has an outer diameter between about 1 mm (3 Fr) to about 11.3 mm (34 Fr).

13. The device of claim 1, wherein the tube body has an inner diameter between about 0.5 mm to about 10 mm.

14. The device of claim 1, wherein the tube body comprises one or more apertures fluidly connected to the at least one lumen.

15. The device of claim 9, wherein the drug-eluting coating comprises a desiccated or hydrated gel composition.

16. The device of claim 9, wherein the drug-eluting coating is a lubricated coating.

17. The device of claim 1, wherein the at least one drug comprises one or more anesthetic selected from the group consisting of: articaine, benzocaine, benzonatate, bupivacaine, chloroprocaine, cinchocaine, diclofenac-diethylamine, dimethocaine, eucaine, etidocaine, exparel, hexylcaine, levobupivacaine, lidocaine, mepivacaine, meprylcaine, oxybuprocaine, phenacaine, piperocaine, pramocaine, prilocaine, procaine, proparacaine, quinisocaine, ropivacaine, tetracaine, and trimecaine.

18. The device of claim 1, wherein the at least one drug comprises one or more adjuvant selected from the group consisting of: epinephrine, narcotics, steroids, clonidine, dexmedetomidine, parecoxib, lornoxicam, midazolam, ketamine, magnesium sulfate, neostigmine, tonicaine, n-butyl tetracaine, adenosine, dextran, neuromuscular blocker, and butamben.

19. The device of claim 1, wherein the at least one drug comprises a therapeutic selected from the group consisting of: anti-viral agents, anti-bacterial agents, anti-thrombolytics, anti-fibrinolytics, chemotherapeutics, anti-hypertensives, immunotherapeutics, antibiotics, anti-inflammatory agents, pro-inflammatory agents, antiseptics, anesthetics, analgesics, pharmaceutical agents, antibodies, small molecules, peptides, and nucleic acids.

20. The device of claim 1, wherein the tube body further comprises one or more reservoir or track, such that the at least one drug is stored in the one or more reservoir or track.

21. The device of claim 1, further comprising a protective sheath encasing at least a portion of the tube body.

22. The device of claim 10, wherein the at least one flexible region comprises one or more springs or corrugations.

23. The device of claim 10, wherein the at least one flexible region comprises a section of the tube body constructed from a flexible and stretchable membrane.

24. The device of claim 10, wherein the at least one flexible region comprises alternating flexible sections and stiff sections.

25. The device of claim 24, wherein the flexible sections comprise a flexible and stretchable membrane.

26. The device of claim 24, wherein the stiff sections comprise a section of the tube body.

27. The device of claim 24, wherein the flexible sections are supplemented with one or more springs.

28. The device of claim 1, wherein the elongate tube body comprises ultra-high-molecular-weight polyethylene.

29. The device of claim 1, wherein the elongate tube body comprises a flexible and stretchable membrane.

30. The device of claim 29, wherein the flexible and stretchable membrane comprises a silicone.

31. The device of claim 1, wherein the elongate tube body comprises a silicone tube.

32. The device of claim 1, wherein the tube device forms part of a medical device selected from the group consisting of: chest tubes, endotracheal tubes, Foley catheters, surgical drains, prosthetics, orthopedic implants, and breast implants.
